(19) United States
(12) Patent Application Publication
(10) Pub. No.: US 2021/0015349 A1
(43) Pub. Date: Jan. 21, 2021

(54) MEDICAL DEVICE

(71) Applicant: OLYMPUS CORPORATION, Tokyo (JP)

(72) Inventor: Tsutomu OKADA, Tokyo (JP)

(73) Assignee: OLYMPUS CORPORATION, Tokyo (JP)

(21) Appl. No.: 17/063,006

(22) Filed: Oct. 5, 2020

Related U.S. Application Data
(63) Continuation of application No. PCT/JP2018/016113, filed on Apr. 19, 2018.

(51) Int. Cl. A61B 1/018; A61B 1/005

(52) U.S. Cl. CPC A61B 1/018; A61B 1/0051

(57) ABSTRACT

A medical device includes: a sheath; a first bending portion provided at a distal end side of the sheath; a second bending portion provided between the first bending portion and the sheath; a first wire fixed to a first wire fixing portion provided at a distal end side of the first bending portion and inserted in the sheath toward a proximal end side of the sheath; and a second wire fixed to a second wire fixing portion provided between the first bending portion and the second bending portion and inserted in the sheath toward the proximal end side of the sheath, wherein a part of the first wire positioned in the first bending portion and a part of the first wire positioned in the second bending portion have straightness, and at least a part of the second wire positioned in the second bending portion has straightness.

### CROSS REFERENCE TO RELATED APPLICATIONS

This application is a continuation application based on a PCT Patent Application No. PCT/JP2018/016113, filed on Apr. 19, 2018, the content of which is incorporated herein by reference.

### BACKGROUND

### Technical Field

The present invention relates to a medical device such as an endoscope or a treatment tool including a bending portion and a wire arranged in the bending portion.

### Background Art

Conventionally, as a method of changing the direction of the distal end of a medical device such as an endoscope or a treatment tool, it is known to provide a bending portion at the distal end of the medical device. In a medical device including a bending portion, a pair of wires is arranged inside the bending portion and each of the pair of wires is fixed to a predetermined portion of the bending portion. For example, a user of a medical device bends a bending portion in a predetermined direction by pulling one wire of a pair of wires at hand, and releases the bending of the bending portion by returning the one wire to the position before the pulling while pulling the other wire at hand.

In a medical device such as an endoscope having a plurality of bending directions, it is necessary to provide a plurality of wires to bend the bending portion in a plurality of directions. For this reason, a plurality of wires are arranged in the insertion portion including the distal end portion of the medical device to be inserted into the patient's body, and it is difficult to reduce the diameter of the insertion portion.

Japanese Patent (Granted) Publication No. 5134971 (hereinafter referred to as Patent Document 1) describes an endoscope including a first bending portion and a second bending portion. In an endoscope including a plurality of bending portions, a plurality of first bending wires for bending the first bending portion in a desired direction and a plurality of second bending wires for bending the second bending portion in a desired direction are provided. In a case in which a plurality of bending portions are provided as in the endoscope described in Patent Document 1, a larger number of wires are required, and it becomes more difficult to reduce the diameter of the insertion portion (sheath) in which the wires are arranged.

In connection with such a problem, in Japanese Unexamined Patent Application, First Publication No. 2012-75661 (hereinafter referred to as Patent Document 2), the number of wires is reduced and the diameter of the endoscope is reduced by limiting the bending of the bending portion by the bending operation in a specific direction. In the endoscope described in Patent Document 2, the bending of the bending portion is limited to one direction (for example, the upward direction), and the wire for bending in the other direction is omitted, so that the contents of the insertion portion are reduced and the diameter of the insertion portion is reduced.

Further, PCT International Publication No. WO 2010/082399 (hereinafter referred to as Patent Document 3) describes an endoscopic treatment tool that bends a bending portion by advancing and retracting a single wire.

In the configurations described in Patent Document 2 and Patent Document 3, the bending of the bending portion and the release (straightening) of the bending are not performed using the pair of wires. Therefore, after the bending portion is once bent, even if an attempt is made to cancel the bending, the bending portion may not return to the original state (for example, the straight state before bending). For example, when selectively inserting and pushing a medical device into a luminal tissue having a bifurcation, it is necessary to bend the bending portion so that the distal end of the medical device is directed in the direction of advance of the medical device, which changes sequentially. However, if the bending of the bending portion is not reliably released, at the bifurcation of the luminal tissue, the distal end of the medical device cannot be directed in the advancing direction of the medical device, and thus it will be difficult to introduce the medical device to the target site and continue appropriate treatment.

Further, in Patent Document 2, in order to restore the bending portion to a substantially linear shape, a treatment tool insertion tube having a relatively large diameter and high rigidity is fixed to a portion on the outer peripheral side of the bending portion, or the cross-sectional area of the wire is increased. However, in such a case, the diameter of the insertion portion may not be sufficiently reduced.

### SUMMARY

An embodiment of the present invention provides a medical device such as an endoscope or a treatment tool that can realize a reduction in diameter by bending a bending portion and releasing (straightening) the bending with a single wire, and can reliably release the bending (straighten) of the bending portion.

A medical device includes: a sheath; a first bending portion provided at a distal end side of the sheath; a second bending portion provided between the first bending portion and the sheath; a first wire fixed to a first wire fixing portion provided at a distal end side of the first bending portion and inserted in the sheath toward a proximal end side of the sheath; and a second wire fixed to a second wire fixing portion provided between the first bending portion and the second bending portion and inserted in the sheath toward the proximal end side of the sheath. A part of the first wire positioned in the first bending portion and a part of the first wire positioned in the second bending portion have straightness. At least a part of the second wire positioned in the second bending portion has straightness.

The first wire and the second wire may be composed of a straight wire member having straightness, and a non-straight wire member made of a material different from that of the straight wire member. At least a part of the first wire positioned in the first bending portion and a part of the first wire positioned in the second bending portion may be composed of the straight wire member. At least a part of the second wire positioned in the second bending portion may be composed of the straight wire member.

The first bending portion and the second bending portion may have lower bending rigidity than the first wire fixing portion, the second wire fixing portion, and the sheath.

The second wire may be arranged with a phase shift of 180 degrees around a central axis of the second bending portion with respect to the first wire.

The second wire may be arranged with a phase shift of 90 degrees around a central axis of the second bending portion with respect to the first wire.

The second wire fixing portion may further include an insertion hole through which the first wire is inserted so as to be capable of moving forward and backward.

According to the medical device of the above aspect, the bending of the bending portion and the release of the bending (straightening) are performed with a single wire, so that the diameter is reduced, and it is possible to reliably release (straighten) the bending of the bending portion.

### BRIEF DESCRIPTION OF THE DRAWINGS

FIG. 1 is a view showing an endoscope treatment tool (medical device) according to a first embodiment of the present invention.

FIG. 2A is a partial cross-sectional view of the distal end portion of the treatment tool.

FIG. 2B is a partial cross-sectional view of the distal end portion of the treatment tool in a state in which the bending portion of the treatment tool is bent.

FIG. 3 is a cross-sectional view of the distal end portion of the treatment tool in a state in which the bending portion of the treatment tool is bent.

FIG. 4A is a schematic view showing an operation in a state in which the treatment tool is used.

FIG. 4B is a schematic view showing an operation in a state in which the treatment tool is used.

FIG. 5 is a partial cross-sectional view showing a configuration of the operation portion of the treatment tool.

FIG. 6A is a schematic view showing a process in a state in which the treatment tool is used.

FIG. 6B is a schematic view showing a process in a state in which the treatment tool is used.

FIG. 7 is a cross-sectional view showing a modification of the configuration of a wire of the treatment tool.

FIG. 8A is a partial cross-sectional view showing a modification of the configuration of the bending portion of the treatment tool.

FIG. 8B is a partial cross-sectional view showing a modification of the configuration of the bending portion of the treatment tool.

FIG. 9 is a view showing an endoscope treatment tool according to a second embodiment of the present invention.

FIG. 10 is a partial cross-sectional view of the distal end portion of the treatment tool.

FIG. 11 is a cross-sectional view of the distal end portion of the treatment tool.

FIG. 12 is a view showing the distal end portion of the treatment tool in a state in which the bending portion of the treatment tool is bent.

FIG. 13 is a cross-sectional view showing a modification of the configuration of a wire of the treatment tool.

FIG. 14 is a partial cross-sectional view of the endoscopic treatment tool according to a third embodiment of the present invention.

FIG. 15 is a cross-sectional view of the distal end portion of the treatment tool.

FIG. 16 is a view showing the distal end portion of the treatment tool in a state in which the bending portion of the treatment tool is bent.

FIG. 17A is a schematic view showing a distal end surface of the endoscope according to a first procedure of the present invention.

FIG. 17B is a schematic view showing a process of the first procedure of the present invention.

FIG. 17C is a schematic view showing a process of the first procedure.

FIG. 17D is a schematic view showing a process of the first procedure.

FIG. 17E is a schematic view showing a process of the first procedure.

FIG. 17F is a schematic view showing a process of the first procedure.

FIG. 17G is a schematic view showing a process of the first procedure.

FIG. 17H is a schematic view showing a process of the first procedure.

FIG. 17I is a schematic view showing a process of the first procedure.

FIG. 17J is a schematic view showing a process of the first procedure.

FIG. 17K is a schematic view showing a process of the first procedure.

FIG. 17L is a schematic view showing a process of the first procedure.

FIG. 17M is a schematic view showing a process of the first procedure.

FIG. 18A is a schematic view showing a process of a modification of the first procedure.

FIG. 18B is a schematic view showing a process of a modification of the first procedure.

FIG. 18C is a schematic view showing a process of a modification of the first procedure.

FIG. 18D is a schematic view showing a process of a modification of the first procedure.

FIG. 19 is a schematic view showing a process of a modification of the first procedure.

FIG. 20 is a view showing an endoscope treatment tool according to a fourth embodiment of the present invention.

FIG. 21A is a view showing a partial cross-section of the distal end portion of the treatment tool.

FIG. 21B is a cross-sectional view taken along the line XXIB-XXIB of FIG. 21A.

FIG. 21C is a cross-sectional view taken along the line XXIC-XXIC of FIG. 21A.

FIG. 22 is a view showing the distal end portion of the treatment tool in a state in which the bending portion of the treatment tool is bent.

FIG. 23A is a schematic view showing a process of a second procedure of the present invention.

FIG. 23B is a schematic view showing a process of the second procedure of the present invention.

FIG. 24A is a schematic view showing a process of the second procedure.

FIG. 24B is a schematic view showing a process of the second procedure.

FIG. 25A is a schematic view showing a process of the second procedure.

FIG. 25B is a schematic view showing a process of the second procedure.

FIG. 26A is a schematic view showing a process of the second procedure.

FIG. 26B is a schematic view showing a process of the second procedure.

FIG. 27A is a schematic view showing a process of the second procedure.

FIG. 27B is a schematic view showing a process of the second procedure.

FIG. 28 is a view showing the straightness of a wire according to the present invention.

FIG. 29 is a view showing the straightness of a wire according to the present invention.

### DETAILED DESCRIPTION OF THE PREFERRED EMBODIMENTS

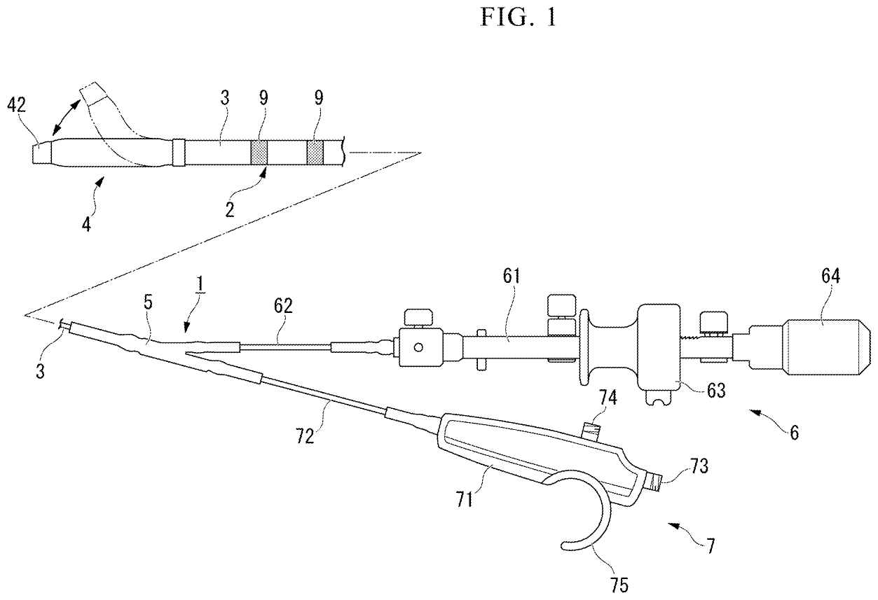

A first embodiment of the present invention will be described with reference to FIGS. 1 to 5. FIG. 1 is a view showing an endoscopic treatment tool 1 (medical device) according to the first embodiment of the present invention. The endoscopic treatment tool 1 (medical device) in the present embodiment is a catheter.

As shown in FIG. 1, an endoscope treatment tool 1 is provided with a long insertion portion 2 extending in the axial direction thereof. The insertion portion 2 constitutes the distal end portion of the endoscopic treatment tool 1. The insertion portion 2 has flexibility and is formed in a tubular shape. The insertion portion 2 is a portion that is inserted into the patient's body when a treatment using the endoscopic treatment tool 1 is performed.

The insertion portion 2 includes a flexible sheath 3 and a bending portion 4 capable of bending. The distal end portion of the insertion portion 2 is configured to include a region at more distal end side than the bending portion 4 and a region of the distal end portion of the bending portion 4. The sheath 3 is configured to have a high rotation followability, and is formed of, for example, a resin tube. The bending portion 4 is provided at more distal end side than the distal end portion of the sheath 3. A marker 9 is provided at a predetermined position on the sheath 3. The marker 9 is formed of a metal material such as stainless steel or platinum, and the position of the insertion portion 2 inserted into the patient's body can be confirmed by grasping the position of the marker 9 under fluoroscopy. Although two markers 9 are provided in FIG. 1, the number thereof may be any number.

Note that the distal end portion of the insertion portion 2 in FIG. 1 is enlarged and shown in order to clearly show the configuration thereof.

The proximal end of the sheath 3 is connected to the connector 5. The connector 5 is connected to a first operation portion 6 arranged to branch laterally from the axis of the sheath 3 and a second operation portion 7 arranged along the axis of the sheath 3.

The first operation portion 6 includes a connecting pipe 62 having one end connected to the connector 5, a first operation portion main body 61 connected to the other end of the connecting pipe 62, a grip 64 fixed to the proximal end portion of the first operation portion main body 61, and a slider 63 mounted so as to be capable of moving forward and backward in the axial direction of the first operation portion body 61.

The second operation portion 7 includes a connecting pipe 72 having one end connected to the connector 5, a second operation portion main body 71 connected to the other end of the connecting pipe 72, a first opening 73 opened in the axial direction of the connecting pipe 72 at the proximal end portion of the second operation portion main body 71, and a second opening 74 opened in a direction substantially orthogonal to the axial direction of the connecting pipe 72 at a more distal end side than the proximal end portion of the second operation portion main body 71. Further, the second operation portion main body 71 is provided with a hook 75 having elasticity and formed in a substantially C shape. The hook 75 can function as a hook for locking the second operation portion body 71 at an appropriate position such as the outer surface of the endoscope.

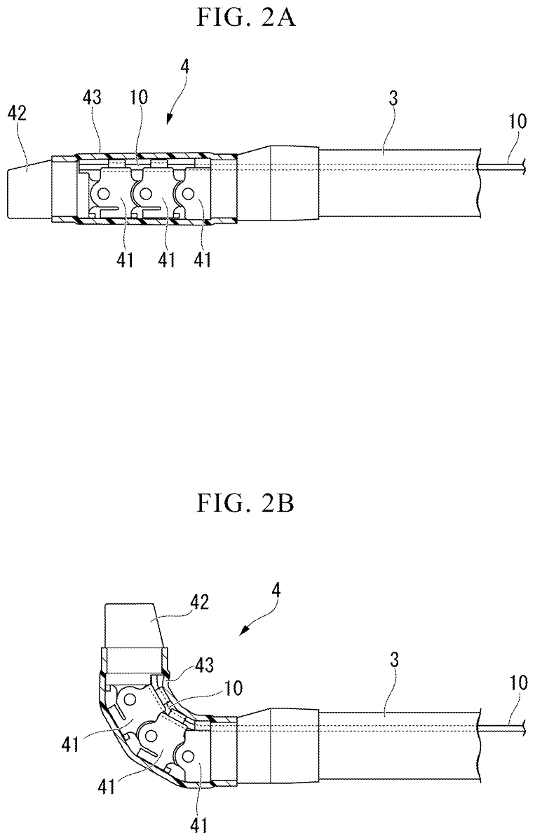

FIG. 2A is a partial cross-sectional view of the distal end portion of the endoscopic treatment tool 1. FIG. 2B is a partial cross-sectional view of the distal end portion of the endoscope treatment tool 1 in a state in which the bending portion 4 of the endoscope treatment tool 1 is bent.

As shown in FIGS. 2A and 2B, the bending portion 4 provided on the distal end side of the distal end portion of the sheath 3 has a plurality of joint pieces 41 that are swingably connected to each other. A wire fixing portion 42 is provided on a more distal end side than the plurality of joint pieces 41. The bending portion 4 is provided at least at a more proximal end side than the wire fixing portion 42. A wire 10 to be described later is fixed to the wire fixing portion 42. The bending portion 4 is formed by covering the plurality of joint pieces 41 with an outer layer 43 having elasticity. Further, a part of the wire fixing portion 42 is covered with the outer layer 43.

In the bending portion 4 of the present embodiment, three joint pieces 41 are connected along the axial direction of the bending portion 4. The number of joint pieces 41 may be appropriately changed according to the curved shape of the bending portion 4.

The maximum outer diameter of the bending portion 4 is preferably an outer diameter that does not extremely expand the inner diameter of the luminal tissue to be inserted. As will be described later, in the present embodiment, since only one wire 10 is arranged in the bending portion 4, the bending portion 4 of the present embodiment has a reduced diameter.

The wire 10 is inserted through the bending portion 4. The distal end of the wire 10 is fixed to the wire fixing portion 42. The wire fixing portion 42 may be provided inside the bending portion 4 (the distal end portion of the bending portion 4). That is, the wire 10 may be fixed to the distal end of the bending portion 4, and the distal end of the bending portion 4 may be used as the wire fixing portion.

The wire 10 extends from the wire fixing portion 42 through the bending portion 4 and the sheath 3 toward the proximal end side. The proximal end portion of the wire 10 is connected to the slider 63 (see FIG. 1) of the first operation portion 6. In the present embodiment, the wire 10 is provided for the bending portion 4, and the number of wires and the number of bending portions are the same.

In a state in which the wire 10 is inserted into the sheath 3 made of a resin tube in the present embodiment and the sheath 3 is in a curved state, the wire 10 has such a straightness that it deforms the sheath 3 from the curved state to a linear state by its own restoring force. Specifically, the wire 10 is formed of a superelastic alloy such as nickel titanium or a linearly processed stainless steel (stainless steel wire such as SUS304-WPBS or SUS304-WPDS).

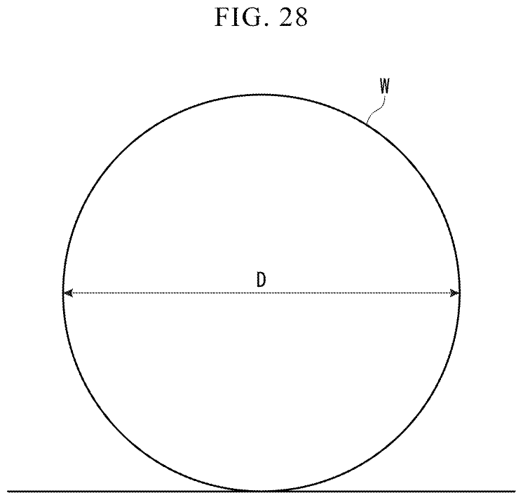

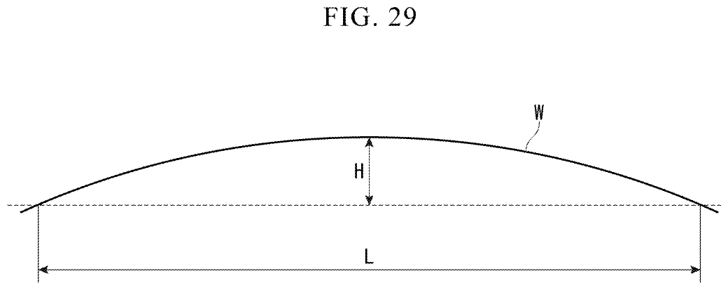

The wire 10 in the present embodiment is a wire having a straightness that can be restored to a state where the length L of the wire W=100 mm and the height H of the arc is H≤mm as shown in FIG. 29, in a state in which the wire W, which has been rounded into a circular shape as shown in FIG. 28, is placed on a table or the like without applying an external force. Note that in a state in which the wire W is rounded as shown in FIG. 28, only the force necessary for rounding the wire W is applied, and the wire W is not strongly bent. The inner diameter D of the circle in FIG. 28 is, for example, about 300 mm.

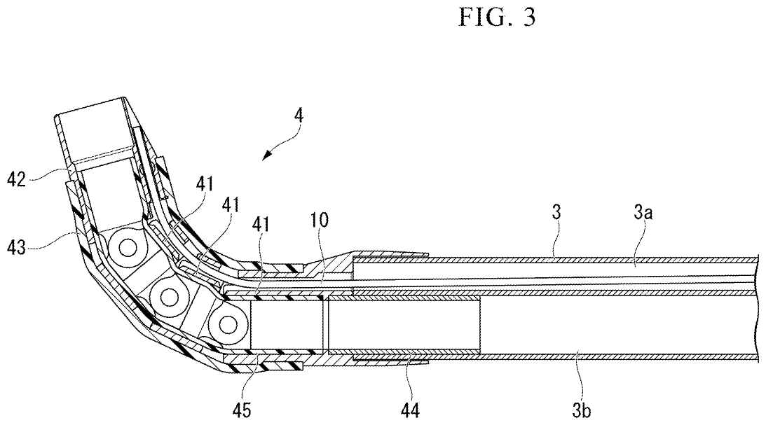

FIG. 3 is a cross-sectional view of the distal end portion of the endoscopic treatment tool 1 in a state in which the bending portion 4 of the endoscopic treatment tool 1 is bent.

As shown in FIG. 3, the sheath 3 has two conduits, a first conduit 3a and a second conduit 3b, inside thereof. The wire 10 is inserted through the first conduit 3a. A connecting pipe 44 is inserted and fixed to the second conduit 3b from the distal end side. The joint piece 41 located at the most proximal end side is firmly fixed to the distal end portion of the sheath 3 via a connecting pipe 44. Each of the plurality of joint pieces 41 and the wire fixing portion 42 is combined so as to form a tubular body in which holes are formed substantially coaxially. The hole of the tubular body communicates with the opening of the wire fixing portion 42. A flexible tube-shaped flexible tube 45 is inserted and fixed in the hole of the cylindrical body. As shown in FIG. 3, the flexible tube 45 covers and closes the gap of the joint piece 41 and the like. For example, by making the flexible tube 45 contain a fluorine compound or the like, or by making the flexible tube 45 made of a fluororesin, it is possible to reduce the sliding resistance between the inner wall of the flexible tube 45 and the insertion object inserted into the flexible tube 45.

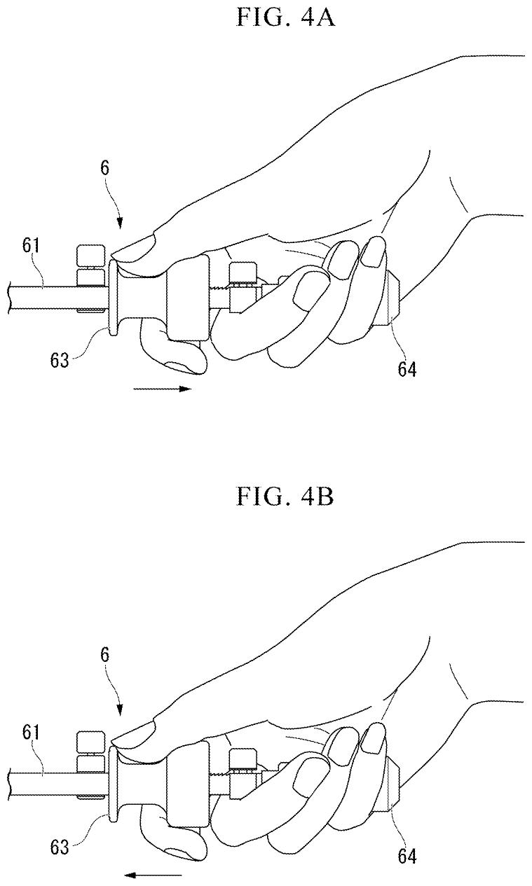

FIG. 4A and FIG. 4B are schematic views showing an operation in a state in which the endoscopic treatment tool 1 is used.

As shown in FIG. 4A, when the user moves the slider 63 to the proximal end side while holding the grip 64 of the first operation portion 6, the wire 10 is pulled along the axial direction of the bending portion 4, and the wire fixing portion 42 is pulled toward the proximal end side. The joint piece 41 is swung in response to the pulling of the wire fixing portion 42, and the bending portion 4 is bent as shown in FIGS. 2B and 3.

In a state in which the bending portion 4 is bent, when the user moves the slider 63 to the distal end side while holding the grip 64 of the first operation portion 6 as shown in FIG. 4B, the traction by the wire 10 is released and the bending portion 4 returns to the straight state (straightened) before being bent as shown in FIG. 2A. Since the wire 10 has straightness, the bending of the bending portion 4 is reliably released (straightened) by the straightness of the wire 10.

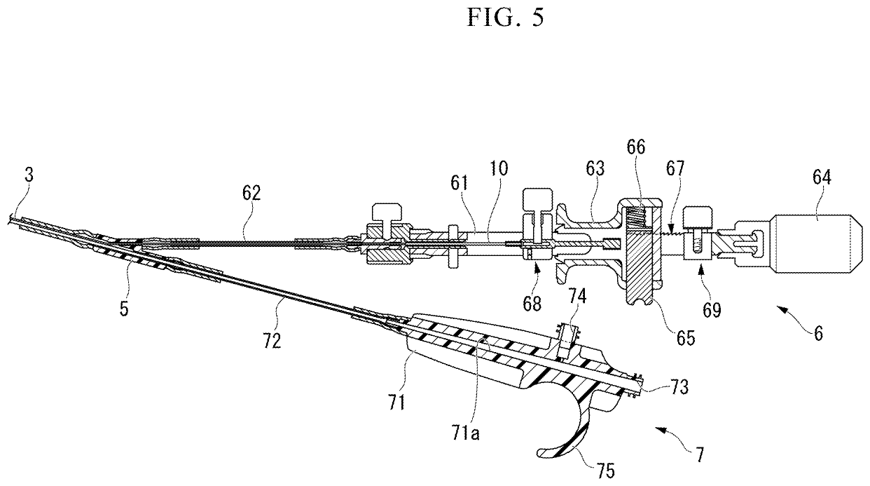

FIG. 5 is a view showing the configuration of the first operation portion 6 and the second operation portion 7 of the endoscope treatment tool 1 in a partial cross section.

As shown in FIG. 5, the first conduit 3a (see FIG. 3) formed in the sheath 3 is connected to the connecting pipe 62 at the connector 5 and communicates with the conduit inside the connecting pipe 62. The wire 10 passes through the wire fixing portion 42, the inside of the bending portion 4, the first conduit 3a inside the sheath 3, and the inside of the connecting pipe 62, and is fixed to the slider 63 via the wire fixing member 68. The slider 63 is provided with a push button 65 and a biasing member 66 that pushes the push button 65 against the first operation portion main body 61. On the outer surface of the first operation portion main body 61, a ratchet portion 67 having a plurality of irregularities is formed at a position where the push button 65 contacts. When the user of the endoscopic treatment tool 1 presses the push button 65, the engagement between the push button 65 and the ratchet portion 67 is released, and the slider 63 can move along the axial direction of the first operation portion main body 61. The user moves the slider 63 to a desired position in the axial direction of the first operation portion main body 61 while keeping the push button 65 pressed. Then, when the user releases the pressing of the push button 65, the push button 65 is pressed against the ratchet portion 67 by the biasing member 66, and the push button 65 and the ratchet portion 67 are engaged with each other. The push button 65 and the ratchet portion 67 are engaged with each other, so that the slider 63 is fixed to a desired position in the axial direction of the first operation portion main body 61. Further, the first operation portion main body 61 is provided with a stopper 69 which the slider 63 can contact. The stopper 69 is provided at more proximal end side than the slider 63. The stopper 69 is configured to be capable of restricting the moving range of the slider 63 in the axial direction.

As shown in FIG. 5, the second conduit 3b (see FIG. 3) formed in the sheath 3 is connected to the connecting pipe 72 at the connector 5 and communicates with the conduit inside the connecting pipe 72. The second operation portion main body 71 is formed with a hole 71a that extends in the axial direction and communicates with the first opening 73 and the second opening 74. The hole 71a communicates with the conduit in the connecting pipe 72. The outer surfaces of the first opening 73 and the second opening 74 are configured to be connectable to a syringe or the like having a known Luer lock mechanism.

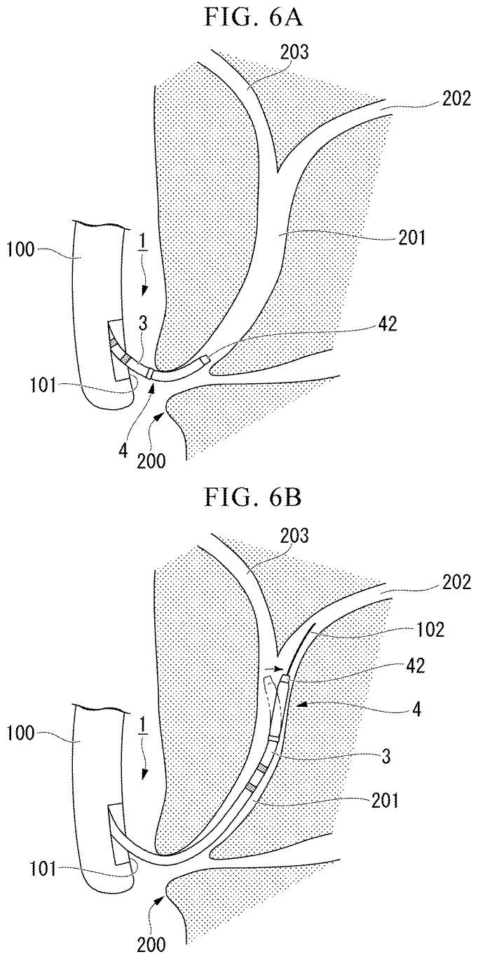

The operation of the endoscopic treatment tool 1 of the present embodiment configured as described above when in use will be described with reference to FIGS. 6A and 6B. Hereinafter, a procedure for inserting a guide wire into the left hepatic duct using the endoscopic treatment tool 1 will be described as an example.

FIG. 6A is a schematic view showing a process of using the endoscopic treatment tool 1, and shows the vicinity of the duodenal papilla 200. The user inserts the endoscope 100 into the body cavity through a natural opening of the patient. In the present embodiment, a side-view endoscope is used as the endoscope 100. The endoscope 100 is provided with a treatment portion 101 for observing a treatment target and making a treatment tool protrude. The user guides the endoscope 100 to the vicinity of the duodenal papilla 200 and captures the duodenal papilla 200 in the visual field of the endoscope 100.

Then, the user inserts the endoscopic treatment tool 1 of the present embodiment into the channel of the endoscope 100. As shown in FIG. 6A, the bending portion 4 and the sheath 3 at the distal end portion of the endoscopic treatment tool 1 are protruded from the treatment portion 101 and inserted into the bile duct 201, which is a luminal tissue, through the opening portion of the duodenal papilla 200. At this time, as shown in FIG. 4A, the user pulls the slider 63 toward the proximal end side while holding the grip 64 of the first operation portion 6, pulls the wire 10, and bends the bending portion 4. By this operation of the user, the distal end portion (wire fixing portion 42) of the endoscopic treatment tool 1 is directed to the bile duct 201 side, which is the traveling direction (insertion direction). Therefore, as shown in FIG. 6A, the user can selectively insert the endoscopic treatment tool 1 into the bile duct 201.

Then, the user pushes the endoscopic treatment tool 1 to the position just before the branch point between the left hepatic duct 202 and the right hepatic duct 203.

FIG. 6B is a schematic view showing a process in a state in which the endoscopic treatment tool 1 is used, and shows a state in which the endoscopic treatment tool 1 is inserted up to just before the branch portion between the left hepatic duct 202 and the right hepatic duct 203. At this time, as shown in FIG. 4B, the user moves the slider 63 to the distal end side while holding the grip 64 of the first operation portion 6, releases the traction by the wire 10, and releases (straightens) the bending of the bending portion 4. Since the wire 10 has straightness, the bending of the bending portion 4 is reliably released by the straightness of the wire 10, and the bending portion 4 is in the straight state before bending. Therefore, as shown in FIG. 6B, the distal end portion (wire fixing portion 42) of the endoscopic treatment tool 1 is directed toward the left hepatic duct 202 side into which a guide wire 102 described later is inserted.

Then, the user inserts the flexible guide wire 102 through the first opening 73 (see FIG. 5) of the second operation portion 7. The guide wire 102 is inserted to the distal end portion (wire fixing portion 42) of the endoscopic treatment tool 1 via the connection tube 72 and the second conduit 3b (see FIG. 3). In the present embodiment, for example, as shown in FIG. 1, the connection tube 72 is arranged along the axis of the sheath 3, so that the insertion resistance of the guide wire 102 is reduced. Therefore, the user can easily insert the guide wire 102 and can appropriately sense the guide wire 102 when the guide wire 102 is inserted into the body cavity.

As shown in FIG. 6B, the user causes the guide wire 102 to protrude from the distal end portion (wire fixing portion 42) of the endoscopic treatment tool 1. At this time, since the distal end portion (wire fixing portion 42) of the endoscopic treatment tool 1 is directed toward the left hepatic duct 202 side, the user can easily and reliably insert the guide wire 102 into the left hepatic duct 202.

As described above, according to the endoscopic treatment tool 1 (medical device) of the present embodiment, the bending of the bending portion 4 and the cancellation (straightening) of the bending are performed by the single wire 10. Therefore, the contents of the endoscopic treatment tool 1 can be reduced and the diameter thereof can be reduced.

Furthermore, according to the endoscopic treatment tool 1 (medical device) of the present embodiment, since the wire 10 has straightness, the bending of the bending portion 4 is reliably released (straightened) by the straightness of the wire 10. Therefore, even in a case in which a pair of wires are not used to operate the bending portion 4, for example, at the branch portion of the luminal tissue, the distal end portion of the endoscopic treatment tool 1 can be reliably oriented in the traveling direction (direction in which the site to be treated is located) and it is possible to introduce the endoscopic treatment tool 1 to the treatment target site and to perform an appropriate treatment on the site to be treated by the endoscopic treatment tool 1.

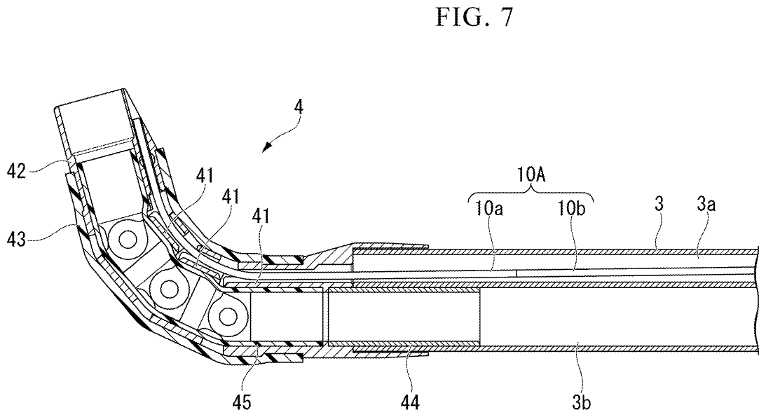

As a modification of the wire configuration of the present embodiment, the wire may be configured by connecting a straight wire member having straightness and a non-straight wire member made of a material different from that of the straight wire member. FIG. 7 is sectional view showing a modification of a configuration of the wire of the endoscope treatment tool 1.

As shown in FIG. 7, the wire 10A according to the present modification includes a straight wire member 10a having straightness and a non-straight wire member 10b made of a material different from that of the straight wire member 10a. Of the wire 10A, at least a portion located inside the bending portion 4 is composed of the straight wire member 10a. The position where the straight wire member 10a and the non-straight wire member 10b are connected is not limited to the position shown in FIG. 7 as long as the position is at a more proximal end side than the proximal end portion of the bending portion 4. Further, from the viewpoint of reducing the diameter of the endoscope treatment tool 1, it is preferable that the connection between the straight wire member 10a and the non-straight wire member 10b be a connection between the end surface of the straight wire member 10a and the end surface of the non-straight wire member 10b.

According to the configuration of the wire 10A of the present modification, the non-straightness wire member 10b does not need to have straightness, and thus an inexpensive member can be used as the non-straightness wire member 10b. Therefore, by adopting the wire 10A, the manufacturing cost of the endoscopic treatment tool 1 can be reduced.

Also in the configuration of the wire 10A of the present modification, the wire 10A is fixed to the wire fixing portion 42, and the portion located inside the bending portion 4 is formed of the straight wire member 10a having straightness. Therefore, the bending of the bending portion 4 is reliably released (straightened) due to the straightness of the wire 10A.

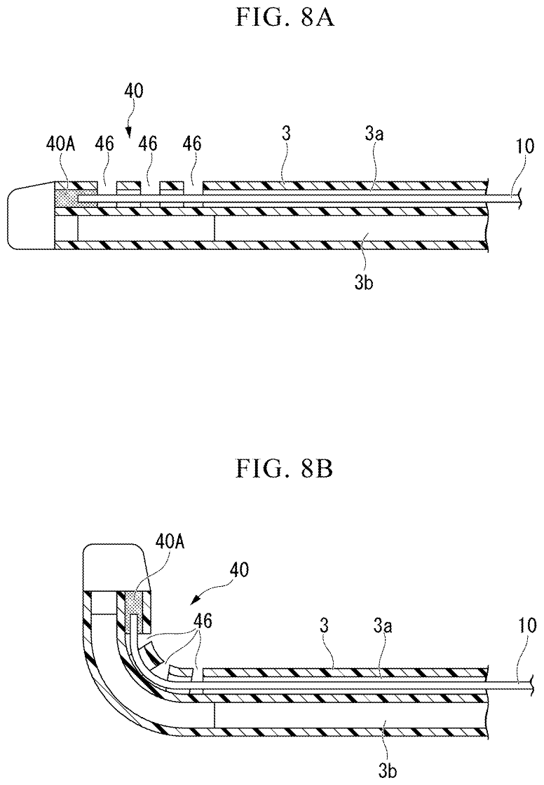

Further, as a modification of the configuration of the bending portion of the present embodiment, the bending portion may be configured by lowering bending rigidity as compared with other portions, instead of providing the joint piece 41. FIGS. 8A and 8B are views showing a modification of the configuration of a bending portion of endoscope treatment tool 1 in a partial section.

As shown in FIG. 8A, the bending portion 40 of the present modification is configured so that the resin tube forming the sheath 3 extends to a more distal end side than the sheath 3 and a notch 46 is provided in a part of the extended resin tube. A wire fixing portion 40A is provided on a more distal end side than the plurality of notches 46, so that the wire 10 is fixed. The portion provided with the notch 46 has lower bending rigidity than the wire fixing portion 40A and the sheath 3. The number, shape, position, and the like of the notches 46 are not limited to the configuration shown in FIG. 8A as long as the bending rigidity of the bending portion 40 can be reduced.

As shown in FIG. 8B, the bending portion 40 can be bent by pulling the wire 10 fixed to the wire fixing portion 40A. In the example shown in FIG. 8B, the bending portion 40 is bent so that the notches 46 approach each other.

As another means for reducing the bending rigidity of the bending portion 40, instead of providing the notch 46, the bending portion 40 may be formed of a material having lower bending rigidity than the resin tube forming the sheath 3.

According to the configuration of the bending portion 40 of the present modification, it is not necessary to provide the joint piece 41 to bend the bending portion 40. Therefore, the configuration of the bending portion 40 can be simplified.

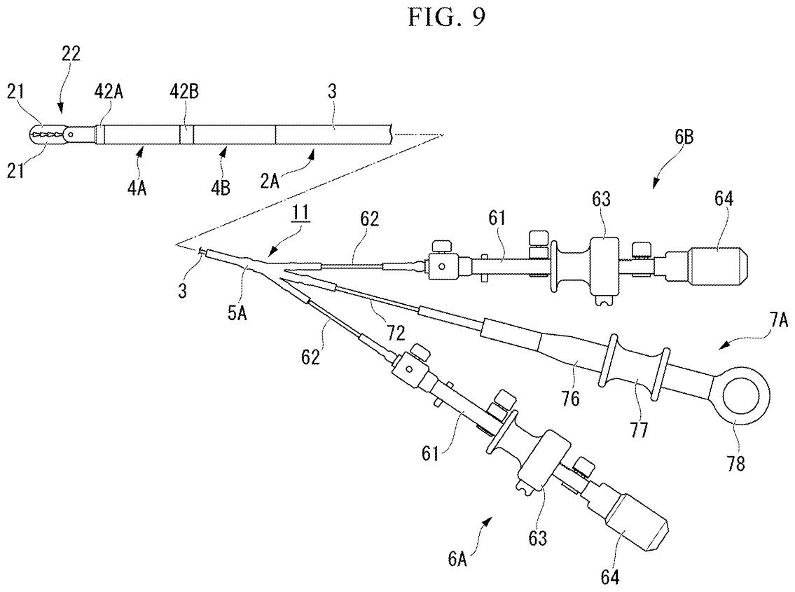

Next, a second embodiment of the present invention will be described with reference to FIGS. 9 to 12. The endoscopic treatment tool (medical device) according to the present embodiment is different from the first embodiment in that it includes at least a plurality of bending portions. In the following description, the same components as those already described will be designated by the same reference numerals and redundant description will be omitted.

FIG. 9 is a view showing the endoscope treatment tool 11 (medical device) according to the second embodiment of the present invention. The endoscopic treatment tool 11 (medical device) in the present embodiment is grasping forceps. The distal end portion of the insertion portion 2A in FIG. 9 is enlarged and shown in order to clearly show the configuration thereof.

As shown in FIG. 9, the endoscopic treatment tool 11 is provided with an insertion portion 2A. The insertion portion 2A includes a sheath 3, a first bending portion 4A (bending portion) that can perform a bending operation, a second bending portion 4B that can perform a bending operation, and a forceps portion 22 having a pair of grasping portions 21 that can be opened and closed. The distal end portion of the insertion portion 2A is configured to include a region at a more distal end side than the first bending portion 4A and a region of the distal end portion of the first bending portion 4A. The first bending portion 4A is provided at a more distal end side than the distal end portion of the sheath 3. The second bending portion 4B is provided between the sheath 3 and the first bending portion 4A. The forceps portion 22 is provided at more distal end side than the first bending portion 4A.

The proximal end of the sheath 3 is connected to the connector 5A. The proximal end side of the connector 5A is branched in three directions, and a first operation portion 6A arranged so as to branch laterally from the axis of the sheath 3, a second operation portion 6B arranged so as to branch laterally from the axis of the sheath 3, and a third operation portion 7A arranged along the axis of the sheath 3 are connected to each other.

The first operation portion 6A is an operation portion for operating the first bending portion 4A, and the second operation portion 6B is an operation portion for operating the second bending portion 4B. The configurations of the first operation portion 6A and the second operation portion 6B are similar to those of the first operation portion 6 of the first embodiment.

The third operation portion 7A is an operation portion for operating the forceps portion 22. The third operation portion 7A includes a connecting pipe 72 one end of which is connected to the connector 5A, a third operation portion main body 76 connected to the other end of the connecting pipe 72, a finger hook ring 78 fixed to the proximal end portion of the third operation portion main body 76, and a slider 77 attached so as to be capable of moving back and forth in the axial direction of the third operation portion main body 76.

FIG. 10 is a view showing the distal end portion of the endoscope treatment tool 11 in a partial cross section.

As shown in FIG. 10, the second bending portion 4B provided at a more distal end side than the distal end portion of the sheath 3 has a plurality of joint pieces 48 swingably connected to each other. A second wire fixing portion 42B is provided at a more distal end side than the plurality of joint pieces 48. The second bending portion 4B is provided at least at a more proximal end side than the second wire fixing portion 42B. The second wire fixing portion 42B is provided between the first bending portion 4A and the second bending portion 4B, and the second wire 26 described later is fixed thereto. The joint piece 48 has a shape such that a gap 48s is formed only on one side in a state in which the plurality of joint pieces 48 are connected to each other. Accordingly, the second bending portion 4B is configured to be capable of bending only on the side where the gap 48s is formed.

The first bending portion 4A provided at a more distal end side than the second wire fixing portion 42B has a plurality of joint pieces 47 that are swingably connected to each other. A first wire fixing portion 42A (wire fixing portion) is provided at a more distal end side than the plurality of joint pieces 47. The first bending portion 4A is provided at least at a more proximal end side than the first wire fixing portion 42A. A first wire 25 (wire) described later is fixed to the first wire fixing portion 42A. The joint piece 47 has a shape such that a gap 47s is formed only on one side in a state in which the plurality of joint pieces 47 are connected to each other. Accordingly, the first bending portion 4A is configured to be capable of bending only on the side where the gap 47s is formed.

In the present embodiment, as shown in FIG. 10, the gap 47s and the gap 48s are arranged so as to face each other. That is, the gap 47s and the gap 48s are arranged with their phases shifted by 180 degrees around the central axis of the insertion portion 2A (around the axis extending in the longitudinal axis direction of the insertion portion 2A). As a result, the bending direction of the first bending portion 4A is opposite to the bending direction of the second bending portion 4B.

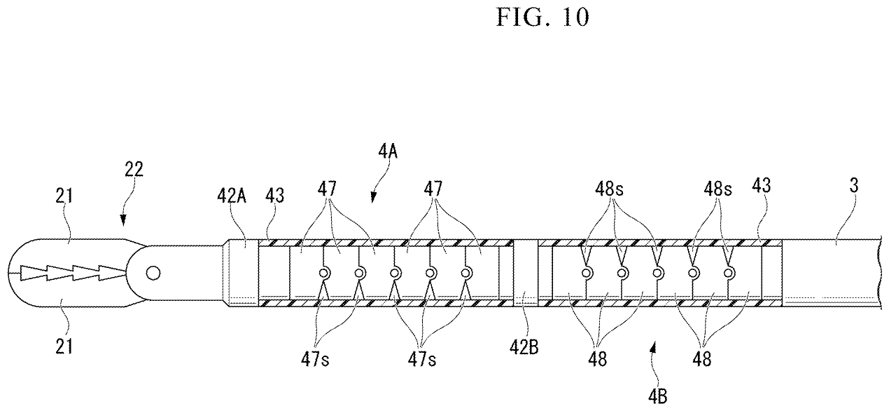

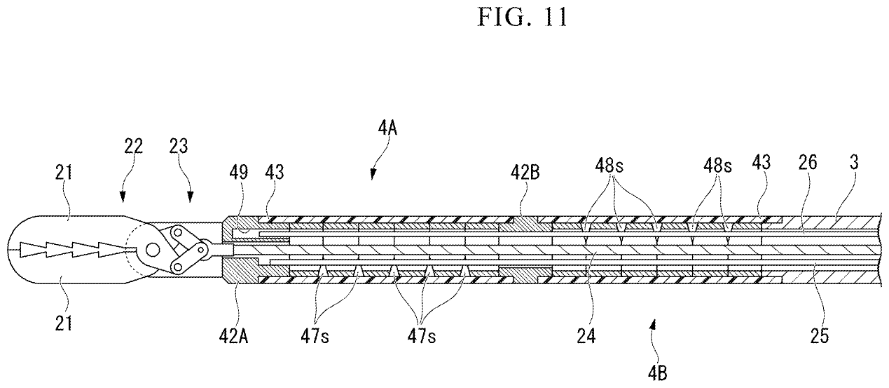

FIG. 11 is a cross-sectional view of the distal end portion of the endoscopic treatment tool 11.

As shown in FIG. 11, the second bending portion 4B is provided at more distal end side than the distal end portion of the sheath 3 and is connected to the distal end portion of the sheath 3. The first bending portion 4A is provided at a more distal end side than the second wire fixing portion 42B and is connected to the second wire fixing portion 42B. The forceps portion 22 is provided at a more distal end side than the first wire fixing portion 42A and is connected to the first wire fixing portion 42A.

The first wire 25 (wire) is inserted into the first bending portion 4A. The first wire 25 is arranged on the side where the gap 47s is formed. The first wire 25 has straightness. The distal end portion of the first wire 25 is fixed to the first wire fixing portion 42A. The first wire fixing portion 42A may be provided inside the first bending portion 4A (the distal end portion of the first bending portion 4A). That is, the first wire 25 may be fixed to the distal end of the first bending portion 4A, and the distal end of the first bending portion 4A may be used as the wire fixing portion. The first wire 25 extends from the first wire fixing portion 42A through the first bending portion 4A, the second bending portion 4B, and the sheath 3 toward the proximal end side. The proximal end portion of the first wire 25 is connected to the slider 63 (see FIG. 9) of the first operation portion 6A.

The second wire 26 is inserted through the second bending portion 4B. The second wire 26 is arranged with a phase shift of 180 degrees with respect to the first wire 25 about the central axis of the second bending portion 4B (about the axis extending in the longitudinal axis direction of the second bending portion 4B), so as to be arranged on the side where the gap 48s is formed. The second wire 26 has straightness. The second wire 26 is fixed to the second wire fixing portion 42B. The second wire fixing portion 42B may be provided inside the second bending portion 4B (the distal end portion of the second bending portion 4B). That is, the second wire 26 may be fixed to the distal end of the second bending portion 4B, and the distal end of the second bending portion 4B may be used as the wire fixing portion. The second wire 26 extends further toward a more distal end side than the fixing point with the second wire fixing portion 42B, and extends to the first wire fixing portion 42A. As shown in FIG. 11, the distal end portion of the second wire 26 is inserted into the insertion hole 49 provided in the first wire fixing portion 42A. The distal end of the second wire 26 is not fixed to the first wire fixing portion 42A, and the distal end of the second wire 26 is inserted into the insertion hole 49 so as to be capable of moving forward and backward. The second wire 26 extends from the first wire fixing portion 42A through the inside of the first bending portion 4A, the inside of the second bending portion 4B, and the inside of the sheath 3 toward the proximal end side. The proximal end portion of the second wire 26 is connected to the slider 63 (see FIG. 9) of the second operation portion 6B.

In the present embodiment, the first wire 25 is provided for the first bending portion 4A, and the second wire 26 is provided for the second bending portion 4B. That is, the number of wires and the number of bending portions are the same.

A link mechanism 23 is provided at the proximal end of the forceps portion 22. A forceps portion operating wire 24 is fixed to the link mechanism 23. The forceps portion operating wire 24 extends from the link mechanism 23 through the first bending portion 4A, the second bending portion 4B, and the sheath 3 toward the proximal end side. The proximal end portion of the forceps portion operating wire 24 is connected to the slider 77 (see FIG. 9) of the third operation portion 7A. When the user moves the slider 77 toward the distal end side along the axial direction of the third operation portion main body 76 while putting his/her finger on the finger hooking ring 78 of the third operation portion 7A, the link mechanism 23 of the forceps portion 22 opens the pair of grasping portions 21. On the contrary, when the user moves the slider 77 to the proximal end side along the axial direction of the third operation portion main body 76, the link mechanism 23 of the forceps portion 22 closes the pair of grasping portions 21.

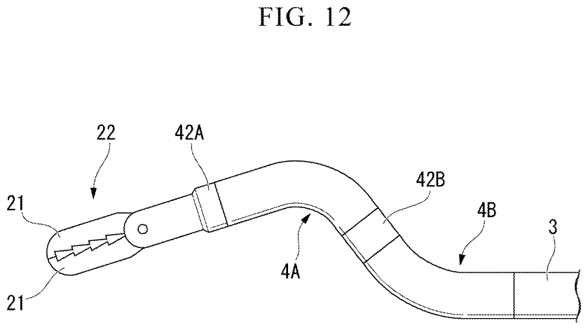

The bending operation of the endoscopic treatment tool 11 of the present embodiment configured as described above will be described with reference to FIG. 12.

FIG. 12 is a view showing the distal end portion of the endoscope treatment tool 11 in a state in which the first bending portion 4A and the second bending portion 4B of the endoscope treatment tool 11 are bent.

When the user moves the slider 63 to the proximal end side while holding the grip 64 of the first operation portion 6A (see FIG. 9), the first wire 25 is pulled along the axial direction of the first bending portion 4A, and the first wire fixing portion 42A is pulled toward the proximal end side. The joint piece 47 swings in response to the pulling of the first wire fixing portion 42A, and the first bending portion 4A is bent to the side where the gap 47s is formed.

When the user moves the slider 63 to the proximal end side while holding the grip 64 of the second operation portion 6B (see FIG. 9), the second wire 26 is pulled along the axial direction of the second bending portion 4B, and the second wire fixing portion 42B is pulled toward the proximal end side. The joint piece 48 swings in response to the pulling of the second wire fixing portion 42B, and the second bending portion 4B is bent to the side where the gap 48s is formed.

In the present embodiment, since the gap 47s and the gap 48s are arranged so as to face each other (the first wire 25 and the second wire 26 face each other), and the bending directions of the first bending portion 4A and the second bending portion 4B are opposite to each other. Therefore, in a state in which both the first bending portion 4A and the second bending portion 4B are bent, the distal end portion of the endoscopic treatment tool 11 has a substantially S-shape as shown in FIG. 12.

In the state where the first bending portion 4A is bent, when the user moves the slider 63 to the distal end side while holding the grip 64 of the first operation portion 6A (see FIG. 9), the traction by the first wire 25 is released, and the first bending portion 4A returns to a straight state before being bent (straightened). Since the first wire 25 has straightness, the straightness of the first wire 25 reliably releases (straightens) the bending of the first bending portion 4A.

In the state where the second bending portion 4B is bent, when the user moves the slider 63 to the distal end side while holding the grip 64 of the second operation portion 6B (see FIG. 9), the traction by the second wire 26 is released, and the second bending portion 4B returns to a straight state before being bent (straightened). Since the second wire 26 has straightness, the straightness of the second wire 26 reliably releases (straightens) the bending of the second bending portion 4B.

Further, in the present embodiment, both the first wire 25 and the second wire 26 extend from the first wire fixing portion 42A through the first bending portion 4A, the second bending portion 4B, and the sheath 3. Thereby, the first bending portion 4A and the second bending portion 4B have substantially the same contents, and it is possible to make the bending rigidity of the first bending portion 4A and the bending rigidity of the second bending portion 4B substantially the same.

As described above, according to the endoscopic treatment tool 11 (medical device) of the present embodiment, the bending of the first bending portion 4A and the release of the bending (straightening) are performed by the single first wire 25. Similarly, the bending of the second bending portion 4B and the release (straightening) of the bending are performed by the single second wire 26. Therefore, even in a case in which a plurality of bending portions are provided, the content of the endoscopic treatment tool 11 can be reduced and the diameter thereof can be reduced.

Further, according to the endoscopic treatment tool 11 (medical device) of the present embodiment, since the first wire 25 and the second wire 26 have straightness, the bending of the bending portion is reliably released (straightened) due to the straightness of the first wire 25 and the second wire 26.

Further, according to the endoscopic treatment tool 11 (medical device) of the present embodiment, since the bending rigidity of the first bending portion 4A and the second bending portion 4B are substantially the same, the release (straightening) of the bending of the first bending portion 4A and the second bending portion 4B can be controlled more appropriately.

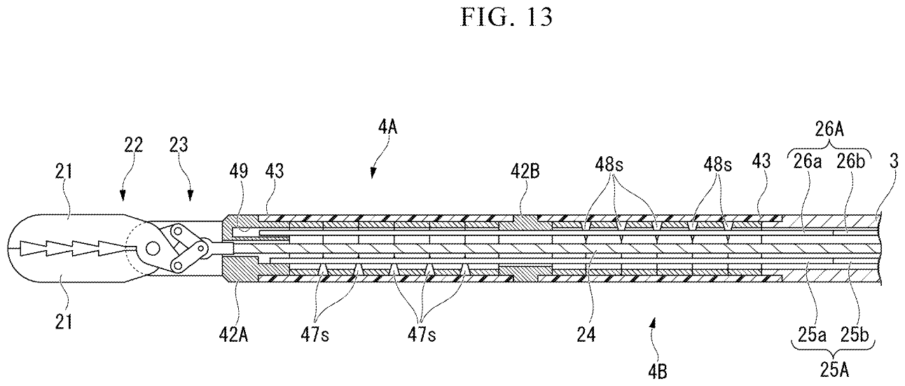

As a modification of the wire configuration of the present embodiment, the wire may be configured by connecting a straight wire member having straightness and a non-straightness wire member made of a material different from the straightness wire member. FIG. 13 is sectional view showing a modification of the structure of the wire of the endoscope treatment tool 11.

As shown in FIG. 13, the first wire 25A of the present modification is composed of a straight wire member 25a having straightness and a non-straight wire member 25b made of a material different from that of the straight wire member 25a. Of the first wire 25A, at least a portion located inside the first bending portion 4A and a portion located inside the second bending portion 4B are composed of the straight wire member 25a. The position where the straight wire member 25a and the non-straight wire member 25b are connected is not limited to the position shown in FIG. 13 as long as the position is more proximal end side than the proximal end portion of the second bending portion 4B. Further, it is preferable from the viewpoint of reducing the diameter that the connection between the straight wire member 25a and the non-straight wire member 25b is a connection between the end surface of the straight wire member 25a and the end surface of the non-straight wire member 25b.

Similarly to the first wire 25A, the second wire 26A may be composed of a straight wire member 26a having straightness and a non-straight wire member 26b made of a material different from that of the straight wire member 26a.

According to the configuration of the first wire 25A and the second wire 26A of the present modification, the non-straight wire member 25b and the non-straight wire member 26b do not need to have straightness, and thus inexpensive members can be used as the non-straight wire member 25b and the non-straight wire member 26b. Therefore, by adopting the first wire 25A and the second wire 26A, it is possible to suppress the manufacturing cost of the endoscopic treatment tool 11.

Also in the configuration of the first wire 25A and the second wire 26A of the present modification, the first wire 25A and the second wire 26A are fixed to the first wire fixing portion 42A and the second wire fixing portion 42B, respectively, and the portion located inside the bending portion is composed of a straight wire member having straightness. Therefore, due to the straightness of the first wire 25A and the second wire 26A, the bending of the first bending portion 4A and the second bending portion 4B is reliably released (straightened).

Further, as a modification of the configuration of the bending portion of the present embodiment, the first bending portion and the second bending portion may be configured to have lower bending rigidity than other portions as the configuration shown in FIGS. 8A and 8B instead of providing the joint pieces. That is, the first bending portion 4A and the second bending portion 4B may be configured to have a lower bending rigidity than the first wire fixing portion 42A, the second wire fixing portion 42B, and the sheath 3.

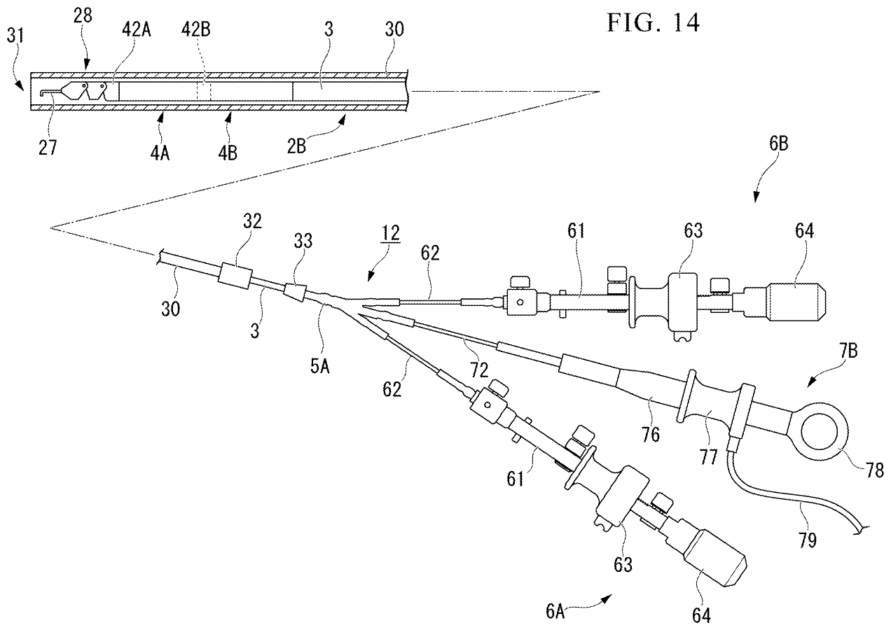

Next, a third embodiment of the present invention will be described with reference to FIGS. 14 to 16. The endoscopic treatment tool (medical device) according to the present embodiment is different from the first embodiment at least in that it includes a plurality of bending portions.

FIG. 14 is a view showing the endoscope treatment tool 12 (medical device) according to the third embodiment of the present invention in partial cross section. The endoscopic treatment tool 12 (medical device) in the present embodiment is a high-frequency knife. Note that the distal end portion of the insertion portion 2B in FIG. 14 is enlarged and shown in order to clearly show the configuration thereof.

As shown in FIG. 14, the endoscopic treatment tool 12 is provided with an insertion portion 2B. The insertion portion 2B includes a sheath 3, a first bending portion 4A (bending portion) capable of performing a bending operation, a second bending portion 4B capable of performing a bending operation, and a swing portion 28 capable of swing. The distal end portion of the insertion portion 2B is configured to include a region on a more distal end side than the first bending portion 4A and a region of the distal end portion of the first bending portion 4A. A hook-shaped electrode 27, for example, is provided at the distal end of the swing portion 28. The swing portion 28 is provided on a more distal end side than the first bending portion 4A.

The insertion portion 2B is provided with a tubular outer sheath tube 30 having electrical insulation and flexibility. The outer sheath tube 30 covers the interior portion 31 including the electrode 27, the swing portion 28, the first bending portion 4A, the second bending portion 4B, and the sheath 3 so as to be able to advance and retract in the axial direction of the insertion portion 2B. An engagement portion 32 is provided at the proximal end portion of the outer sheath tube 30. An engagement body 33 is provided at the proximal end of the sheath 3 (proximal end of the interior portion 31). The engagement portion 32 and the engagement body 33 are configured to be engageable with each other. The engagement portion 32 is configured to engage with the engagement body 33 in a state in which the electrode 27, the swing portion 28, the first bending portion 4A, and the second bending portion 4B completely protrude from the outer sheath tube 30 toward the distal end side. In a state in which the engagement portion 32 and the engagement body 33 are engaged with each other, the outer sheath tube 30 and the interior portion 31 are fixed to each other, and the state in which the interior portion 31 protrudes from the outer sheath tube 30 toward the distal end side is held.

The proximal end of the sheath 3 is connected to the connector 5A via the engagement body 33. The proximal end side of the connector 5A is branched in three directions. The first operation portion 6A arranged so as to be branched laterally from the axis of the sheath 3, the second operation portion 6B arranged so as to be branched laterally from the axis of the sheath 3, and the third operation portion 7B arranged along the axis of the sheath 3 are connected to each other.

The first operation portion 6A is an operation portion for operating the first bending portion 4A, and the second operation portion 6B is an operation portion for operating the second bending portion 4B. The configurations of the first operation portion 6A and the second operation portion 6B are similar to those of the first operation portion 6 of the first embodiment.

The third operation portion 7B is an operation portion for operating the electrode 27 and the swing portion 28. The configuration of the third operation portion 7B is the same as that of the third operation portion 7A of the second embodiment, but in the third operation portion 7B of the present embodiment, a power cord 79 for supplying a high frequency current to the electrode 27 is connected to the slider 77.

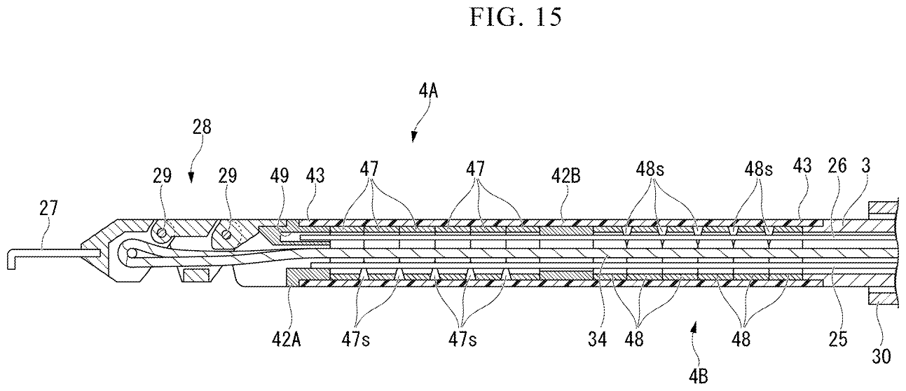

FIG. 15 is a cross-sectional view of the distal end portion of the endoscopic treatment tool 12.

As shown in FIG. 15, the second bending portion 4B is provided at a more distal end side than the distal end portion of the sheath 3 and is connected to the distal end portion of the sheath 3. The first bending portion 4A is provided at a more distal end side than the second wire fixing portion 42B and is connected to the second wire fixing portion 42B. The swing portion 28 is provided at a more distal end side than the first wire fixing portion 42A (wire fixing portion) and is connected to the first wire fixing portion 42A. An electrode 27 is attached to the distal end of the swing portion 28.

The configurations of the first bending portion 4A, the second bending portion 4B, the first wire 25 (wire), and the second wire 26 are the same as those of the second embodiment, but in the present embodiment, the second wire fixing portion 42B is completely covered by the outer layer 43.

Similar to the second embodiment, the first wire 25 and the second wire 26 have straightness. The first wire 25 is fixed to the first wire fixing portion 42A. The second wire 26 is fixed to the second wire fixing portion 42B, extends further toward a more distal end side than the fixing point, and extends to the first wire fixing portion 42A. As shown in FIG. 15, the distal end portion of the second wire 26 is inserted in an insertion hole 49 provided in the first wire fixing portion 42A so as to be capable of moving forward and backward.

The swing portion 28 has at least one joint 29. In the example shown in FIG. 15, the swing portion 28 has two joints 29, and an electrode swing wire 34 is connected to a more distal end side than the joint 29 closest to the distal end side.

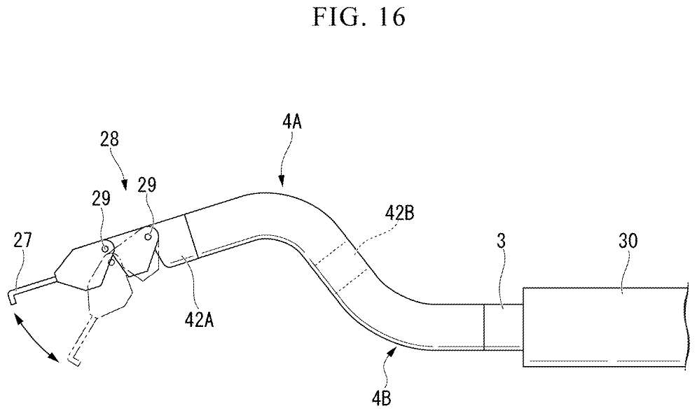

The electrode swing wire 34 extends from the swing portion 28 through the first bending portion 4A, the second bending portion 4B, and the sheath 3 toward the proximal end side. The proximal end portion of the electrode swing wire 34 is connected to the slider 77 (see FIG. 14) of the third operation portion 7B. The user moves the slider 77 forwards and backwards along the axial direction of the third operation portion main body 76 while putting his/her finger on the finger hooking ring 78 of the third operation portion 7B, so that the swing portion 28 swings and the electrode 27 swings. (See FIG. 16). The electrode swing wire 34 is configured to be electrically conductive with the power cord 79.

The bending operation of the endoscopic treatment tool 12 of the present embodiment configured as described above will be described with reference to FIG. 16. FIG. 16 is a view showing the distal end portion of the endoscope treatment tool 12 when the first bending portion 4A and the second bending portion 4B of the endoscope treatment tool 12 are bent.

The bending operation of the first bending portion 4A and the second bending portion 4B is the same as in the second embodiment. Also in the present embodiment, as shown in FIG. 15, since the gap 47s and the gap 48s are arranged to face each other (the first wire 25 and the second wire 26 face each other), the bending directions of the first bending portion 4A and the second bending portion 4B are opposite to each other. Therefore, in a state in which both the first bending portion 4A and the second bending portion 4B are bent, the distal end portion of the endoscopic treatment tool 12 has a substantially S-shape as shown in FIG. 16.

Also in the present embodiment, since the first wire 25 has straightness, when returning the first bending portion 4A to the straight state before bending, the bending of the first bending portion 4A is reliably released (straightened) due to the straightness of the first wire 25. Similarly, since the second wire 26 has straightness, when the second bending portion 4B is returned to the straight state before bending, the bending of the second bending portion 4B is reliably released (straightened) due to the straightness of the second wire 26.

Further, also in the present embodiment, both the first wire 25 and the second wire 26 extend from the first wire fixing portion 42A through the inside of the first bending portion 4A, the inside of the second bending portion 4B, and the inside of the sheath 3. Thereby, the first bending portion 4A and the second bending portion 4B have substantially the same contents, and the bending rigidity of the first bending portion 4A and the bending rigidity of the second bending portion 4B can be made substantially the same.

As described above, also in the endoscopic treatment tool 12 (medical device) of the present embodiment, as in the second embodiment, even in a state in which a plurality of bending portions are provided, it is possible to reduce the content of the endoscope treatment tool 12 and realize a reduction in diameter.

Further, also in the endoscope treatment tool 12 (medical device) of the present embodiment, since the first wire 25 and the second wire 26 have straightness, the bending of the bending portion is reliably released (straightened) due to the straightness of the first wire 25 and the second wire 26.

Further, also in the endoscopic treatment tool 12 (medical device) of the present embodiment, since the bending rigidity of the first bending portion 4A and the second bending portion 4B are substantially the same, the release (straightening) of the bending of the first bending portion 4A and the second bending portion 4B can be controlled more reliably.

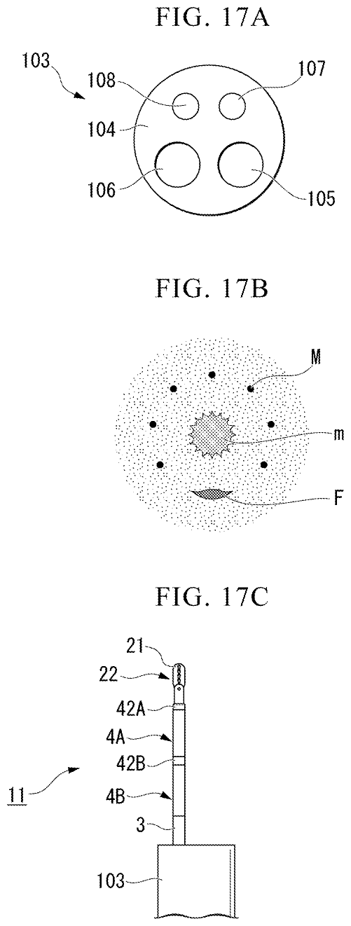

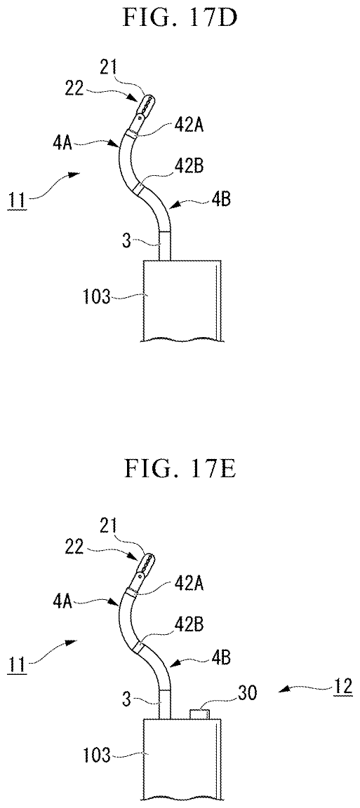

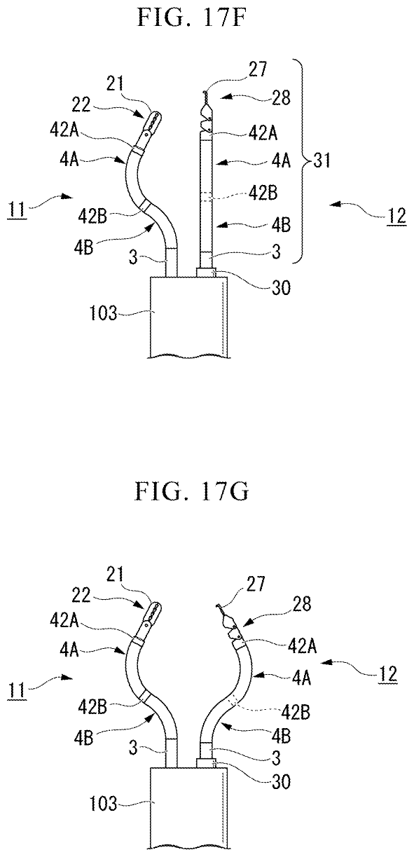

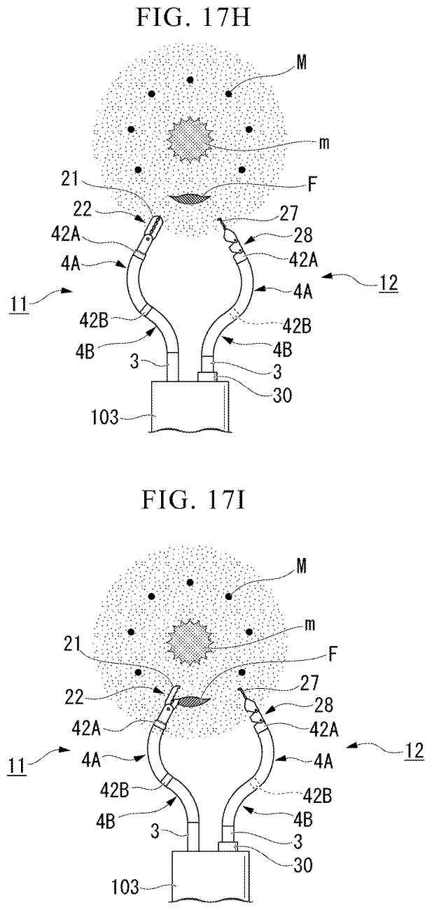

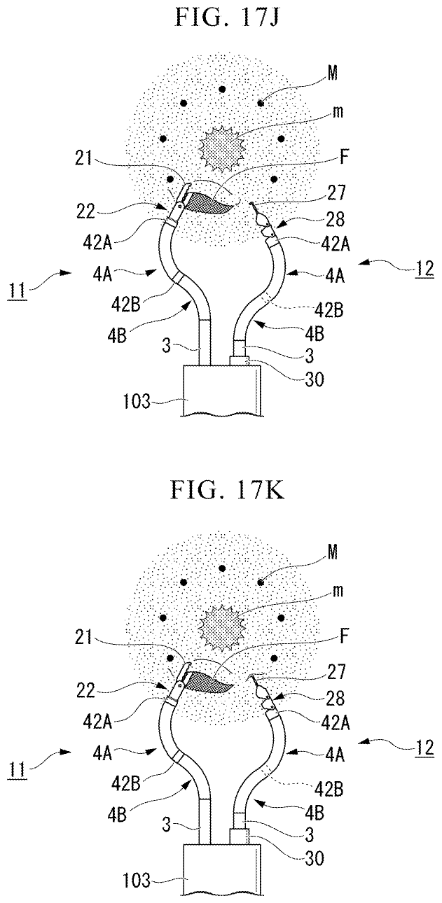

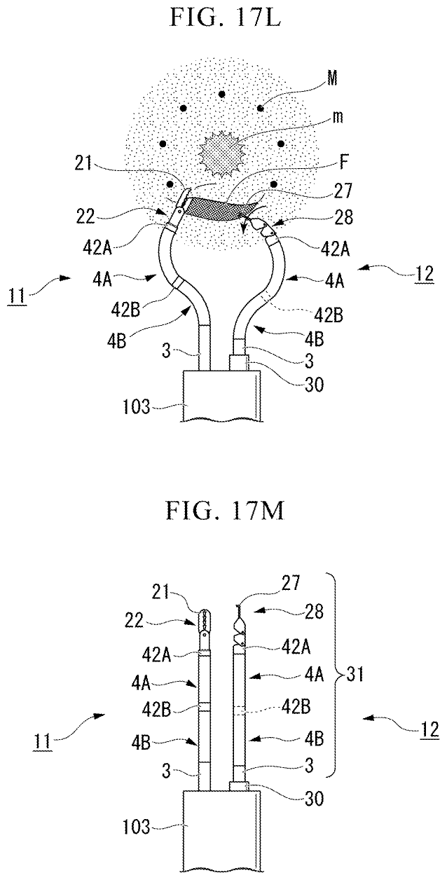

A first procedure using the endoscopic treatment tool 11 (medical device) of the second embodiment and the endoscopic treatment tool 12 (medical device) of the third embodiment will be described with reference to FIGS. 17A to 17M.

FIG. 17A is a schematic view showing the distal end surface 104 of the endoscope 103 according to the first procedure. The endoscope 103 has a treatment tool insertion channel 105, a treatment tool insertion channel 106, an observation portion 107, and an illumination portion 108.

The treatment tool insertion channel 105 and the treatment tool insertion channel 106 are lumens provided in the endoscope 103 for inserting an appropriate treatment tool used for treatment and advancing and retracting from the distal end surface 104 of the endoscope 103. In the first procedure, the endoscopic treatment tool 11 of the second embodiment, which is a grasping forceps, is inserted through the treatment tool insertion channel 105, and the endoscope treatment tool 12 of the third embodiment, which is a high-frequency knife, is inserted through the treatment instrument insertion channel 106.

The observation portion 107 includes an imaging portion including a solid-state imaging device, and an image acquired by the imaging portion is displayed on an external monitor (not shown) or the like connected to the endoscope 103 through the wiring or the like that is passed through the endoscope 103.

The illumination portion 108 has a light source such as a light emitting diode (LED), a light guide connected to the light source, and the like, and illuminates an observation target of the observation portion 107.

The endoscope 103 is not limited to the configuration shown in FIG. 17A as long as it has a plurality of treatment tool insertion channels and is configured to be capable of observing the treatment target.

A case of excising a lesion m in a body cavity, for example, a mucous membrane of a digestive tract by using the endoscope 103 having the treatment tool insertion channel 105 and the treatment tool insertion channel 106 will be described with reference to FIGS. 17B to 17M. FIG. 17B to 17M are schematic views showing a process of the first procedure.

The endoscope 103 is inserted into the body cavity through a natural opening of the patient and is inserted up to the vicinity of the lesion m as the target site. As shown in FIG. 17B, marking M is performed on the mucous membrane of the digestive tract having a lesion m by a known method. Subsequently, physiological saline is injected into the submucosal layer to swell the lesion m, and an initial incision F is made to a part of the mucous membrane around the lesion m.

Next, as shown in FIG. 17C, the endoscopic treatment tool 11 (grip forceps) is inserted into the treatment tool insertion channel 105 of the endoscope 103, and the first bending portion 4A (bending portion) and the second bending portion 4B of the endoscopic treatment tool 11 are protruded from the endoscope 103 toward the distal end side.

Next, as shown in FIG. 17D, the first operation portion 6A (see FIG. 9) of the endoscopic treatment tool 11 is operated to pull the first wire 25 (wire) (see FIG. 11) to bend the first bending portion 4A. Further, the second operation portion 6B (see FIG. 9) of the endoscopic treatment tool 11 is operated to pull the second wire 26 (see FIG. 11) to bend the second bending portion 4B. Due to the bending of the first bending portion 4A and the second bending portion 4B, the distal end portion of the endoscopic treatment tool 11 has a substantially S-shape.

Next, as shown in FIG. 17E, the endoscopic treatment tool 12 (high frequency knife) is inserted into the treatment tool insertion channel 106 of the endoscope 103, and the outer sheath tube 30 of the endoscope treatment tool 12 is protruded from the endoscope 103 toward the distal end side.

Next, as shown in FIG. 17F, the interior portion 31 including the electrode 27, the swing portion 28, the first bending portion 4A, the second bending portion 4B, and the sheath 3 is protruded from the outer sheath tube 30 toward the distal end side. At this time, by engaging the engagement portion 32 and the engagement body 33 (see FIG. 14), the outer sheath tube 30 and the interior portion 31 are fixed to each other.

Next, as shown in FIG. 17G the first operation portion 6A (see FIG. 14) of the endoscopic treatment tool 12 is operated to pull the first wire 25 (wire) (see FIG. 15) to bend the bending portion 4A. Further, the second operation portion 6B (see FIG. 14) of the endoscopic treatment tool 12 is operated to pull the second wire 26 (see FIG. 15) to bend the second bending portion 4B. Due to the bending of the first bending portion 4A and the second bending portion 4B, the distal end portion of the endoscopic treatment tool 12 has a substantially S-shape.

In addition, the endoscopic treatment tool 11 and the endoscopic treatment tool 12 are arranged in the treatment tool insertion channel such that the second wire fixing portions 42B of the endoscopic treatment tool 11 and the endoscopic treatment tool 12 are separated from each other when being bent. That is, the endoscope treatment tool 11 is arranged in the treatment tool insertion channel 105 of the endoscope 103 so that the first wire fixing portion 42A is directed to the inside in the radial direction of the endoscope 103 and the second wire fixing portion 42B is directed to the outside in the radial direction of the endoscope 103 during the bending. The endoscope treatment tool 12 is arranged in the treatment tool insertion channel 106 so that the first wire fixing portion 42A is directed to the inside in the radial direction of the endoscope 103 and the second wire fixing portion 42B is directed to the outside in the radial direction of the endoscope 103 during the bending.

Next, as shown in FIG. 17H, the endoscope treatment tool 11 and the endoscope treatment tool 12 are made to face the lesion m. In the present embodiment, the endoscopic treatment tool 11 and the endoscopic treatment tool 12 are arranged in the treatment tool insertion channel such that the second wire fixing portions 42B of the endoscopic treatment tool 11 and the endoscopic treatment tool 12 are separated from each other when being bent. Therefore, as shown in FIG. 17H, the endoscopic treatment tool 11 and the endoscopic treatment tool 12 can be removed from the center of the visual field of the observation portion 107 of the endoscope 103, and the surgical field including the lesion m can be clearly observed.

Next, as shown in FIG. 17I, the third operation portion 7A (see FIG. 9) of the endoscopic treatment tool 11 is operated to move the forceps portion operating wire 24 (see FIG. 11) forward and backward, and a part of the mucous membrane at the open end of the initial incision F is grasped by the pair of grasping portions 21.

Next, as shown in FIG. 17J, in a state in which a part of the mucous membrane is grasped by the pair of grasping portions 21, the endoscopic treatment tool 11 is pushed further toward the distal end side (the side where the lesion m is located), and tension is applied to the mucous membrane so as to widen the opening of the incision F.

Next, as shown in FIG. 17K, the endoscopic treatment tool 12 is pushed further toward a more distal end side (the side where the lesion m is located) to bring the electrode 27 into contact with the mucous membrane in the vicinity of the initial incision F.

Next, as shown in FIG. 17L, in a state in which the electrode 27 of the endoscopic treatment tool 12 is brought into contact with the mucous membrane tensioned by the endoscopic treatment tool 11, the third operation portion 7B (see FIG. 14) is operated, the electrode swing wire 34 (see FIG. 15) is moved forward and backward, and the swing portion 28 swings and the electrode 27 swings, thereby incising the mucous membrane to excise the mucous membrane including the lesion m. At this time, a high frequency current is supplied to the electrode 27 via the power cord 79 (see FIG. 14).

As the incision of the mucous membrane is advanced, the mucous membrane grasped by the pair of grasping portions 21 of the endoscopic treatment tool 11 may slacken, and the endoscopic treatment tool 12 may be covered. In that case, the endoscopic treatment tool 11 is pushed further toward a more distal end side (the side where the lesion m is located), so that tension is applied to the mucous membrane grasped by the grasping portion 21.

When the excision of the lesion m is completed by repeatedly performing the incision of the mucous membrane, as shown in FIG. 17M, the first operation portion 6A and the second operation portion 6B (see FIG. 9) of the endoscopic treatment tool 11 are operated, the traction by the first wire 25 and the second wire 26 (see FIG. 11) is released, and the first bending portion 4A and the second bending portion 4B of the endoscopic treatment tool 11 are returned to the straight state before bending (straightened). Since the first wire 25 and the second wire 26 have straightness, the bending of the first bending portion 4A and the second bending portion 4B is reliably released (straightened) due to the straightness of the first wire 25 and the second wire 26.

Similarly, the first operation portion 6A and the second operation portion 6B (see FIG. 14) of the endoscopic treatment tool 12 are operated, the traction by the first wire 25 and the second wire 26 (see FIG. 15) is released, and the first bending portion 4A and the second bending portion 4B of the endoscopic treatment tool 12 are returned to the straight state before bending (straightened). Since the first wire 25 and the second wire 26 have straightness, the bending of the first bending portion 4A and the second bending portion 4B is reliably released (straightened) due to the straightness of the first wire 25 and the second wire 26.

Finally, the endoscopic treatment tool 11 and the endoscopic treatment tool 12 are removed from the endoscope 103, and a series of treatments is completed. The endoscopic treatment tool 12 is withdrawn from the endoscope 103 after the interior portion 31 is housed in the outer sheath tube 30 so that the electrode 27 is not caught in the treatment tool insertion channel 106.

As described above, also in the procedure (first procedure) using the endoscopic treatment tool 11 (medical device) and the endoscopic treatment tool 12 (medical device), the first wire 25 and the second wire 26 has straightness. Thereby, it is possible to reliably release (straighten) the bending of the bending portion due to the straightness of the first wire 25 and the second wire 26, and a smooth procedure can be performed. Further, since the bending rigidity of the first bending portion 4A and the second bending portion 4B are substantially the same, the release (straightening) of the bending of the first bending portion 4A and the second bending portion 4B can be controlled more preferably, to carry out the procedure.

As a modification of the first procedure, when the submucosal layer sm is peeled off, the endoscopic treatment tool 11 (grasping forceps) may be arranged in the treatment tool insertion channel 105 by being rotated by 90 degrees, so that the first bending portion 4A bends downward and the second bending portion 4B bends upward.

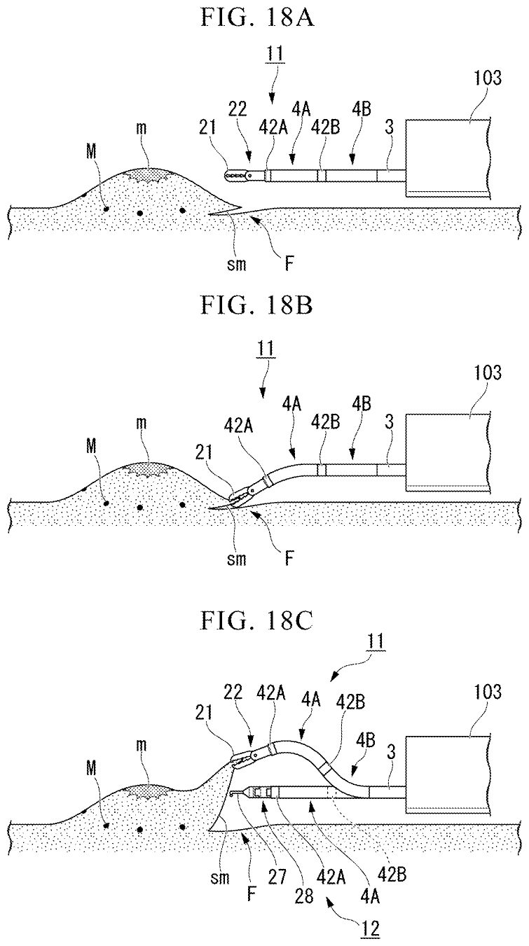

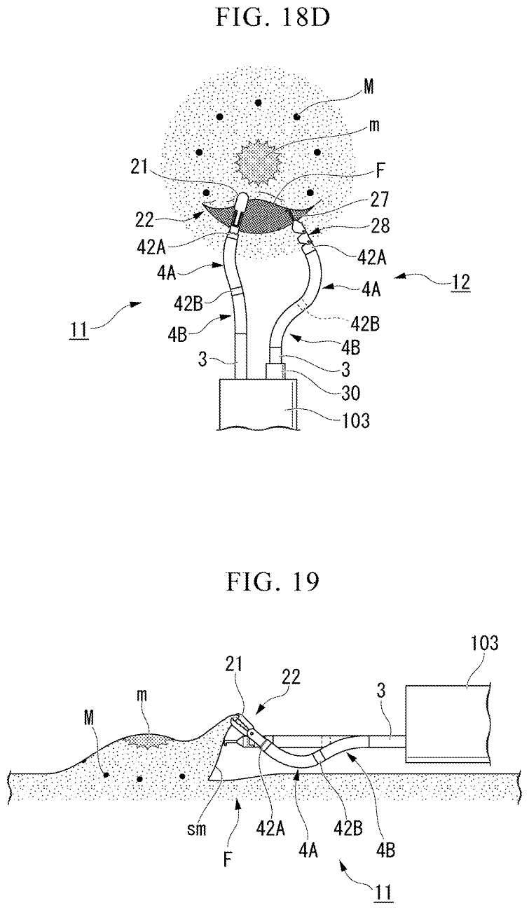

FIGS. 18A to 18D are schematic views showing a process of a modification of the first procedure.

As shown in FIG. 18A, the first bending portion 4A and the second bending portion 4B of the endoscopic treatment tool 11 are protruded from the endoscope 103 toward the distal end side. In the present modification, the endoscopic treatment tool 11 is arranged to be inserted into the treatment tool insertion channel 105 (see FIG. 17A) such that the opening/closing direction of the pair of grasping portions 21 of the forceps portion 22 is the vertical direction. In addition, in the present modification, the endoscopic treatment tool 11 is arranged such that the first bending portion 4A is bent downward and the second bending portion 4B is bent upward.

Note that the bending portion bends downward means that the bending portion bends so that its distal end faces downward. Similarly, the upward bending of the bending portion means that the bending portion bends so that its distal end faces upward.

Next, as shown in FIG. 18B, the first bending portion 4A of the endoscopic treatment tool 11 is bent downward, and a part of the mucous membrane at the open end of the initial incision F is held by the pair of grasping portions 21 of the forceps portion 22.

Next, as shown in FIG. 18C, the second bending portion 4B of the endoscopic treatment tool 11 is bent upward, and the mucous membrane grasped by the pair of grasping portions 21 is pulled upward. Here, similarly to the case of the first procedure described above, the first bending portion 4A and the second bending portion 4B of the endoscopic treatment tool 12 are protruded from the endoscope 103 and the outer sheath tube 30 (see FIG. 18D) toward the distal end side. The arrangement of the endoscope treatment tool 12 in the treatment tool insertion channel 106 (see FIG. 17A) is the same as in the case of the above-described first procedure, and it is arranged so that the swing direction of the electrode 27 of the swing portion 28 is horizontal.

The first bending portion 4A and the second bending portion 4B of the endoscopic treatment tool 12 may be protruded from the endoscope 103 and the outer sheath tube 30 before the grasping portion 21 of the endoscopic treatment tool 11 grasps the mucous membrane. That is, in the process shown in FIG. 18A, the first bending portion 4A and the second bending portion 4B of the endoscopic treatment tool 12 may be protruded from the endoscope 103 and the outer sheath tube 30 toward the distal end side.

Next, as shown in FIG. 18D, with the electrode 27 of the endoscopic treatment tool 12 in contact with the submucosal layer sm, the swing portion 28 is swung to swing the electrode 27 to which high-frequency current is supplied, thereby the submucosal layer sm is incised. In the endoscope treatment tool 12, the electrode 27 is swung by the swing portion 28 to incise the submucosal layer sm, so that the submucosal layer sm can be incised without moving the endoscope 103.

In this modification, the endoscope treatment tool 11 may be arranged in the treatment tool insertion channel 105 (see FIG. 17A) so that the first bending portion 4A bends upward and the second bending portion 4B bends downward.

In this case, as shown in FIG. 19, the second bending portion 4B of the endoscopic treatment tool 11 is bent downward to hold the mucous membrane with the pair of grasping portions 21, and then the first bending portion 4A is bent upward to lift up the grasped mucous membrane. Then, the submucosal layer sm can be incised by the endoscopic treatment tool 12 while the mucous membrane is lifted up by the endoscopic treatment tool 11.

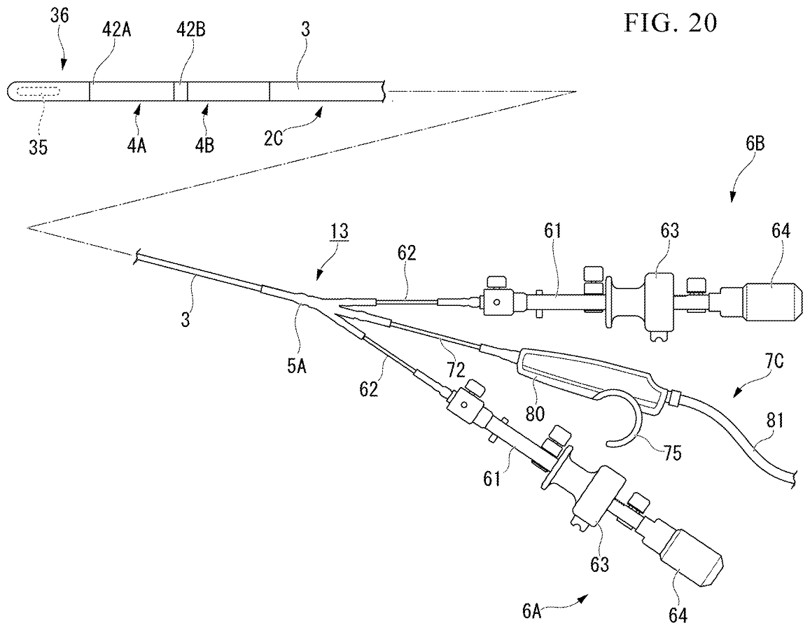

Next, a fourth embodiment of the present invention will be described with reference to FIGS. 20 to 22. The endoscopic treatment tool (medical device) according to the present embodiment is different from the first embodiment at least in that it includes a plurality of bending portions.

FIG. 20 is a view showing an endoscope treatment tool 13 (medical device) according to the fourth embodiment of the present invention. The endoscopic treatment tool 13 (medical device) in the present embodiment is a mucous membrane lifting tool. Note that the distal end portion of the insertion portion 2C in FIG. 20 is enlarged and shown in order to clearly show the configuration thereof.

As shown in FIG. 20, the endoscope treatment tool 13 is provided with an insertion portion 2C. The insertion portion 2C includes a sheath 3, a first bending portion 4A (bending portion) capable of performing a bending operation, a second bending portion 4B capable of performing a bending operation, and a suction portion 36 capable of sucking mucous membranes and the like. The distal end portion of the insertion portion 2C is configured to include a region at a more distal end side than the first bending portion 4A and a region of the distal end of the first bending portion 4A. A suction port 35 is provided on the side of the distal end of the suction portion 36.

The proximal end of the sheath 3 is connected to the connector 5A. The proximal end side of the connector 5A is branched in three directions. The first operation portion 6A arranged so as to be branched laterally from the axis of the sheath 3, the second operation portion 6B arranged so as to be branched laterally from the axis of the sheath 3, and the third operation portion 7C arranged along the axis of the sheath 3 are connected to each other.

The first operation portion 6A is an operation portion for operating the first bending portion 4A, and the second operation portion 6B is an operation portion for operating the second bending portion 4B. The configurations of the first operation portion 6A and the second operation portion 6B are similar to those of the first operation portion 6 of the first embodiment.

A connecting pipe 72, one end of which is connected to the connector 5A, a third operation portion main body 80 connected to the other end of the connecting pipe 72, and a suction tube 81 connected to the third operation portion main body 80 are provided in the third operation portion 7C. The suction tube 81 is connected to a suction device (not shown). Further, the third operation portion main body 80 is provided with a hook 75 for locking the third operation portion main body 80 at an appropriate position such as the outer surface of the endoscope.

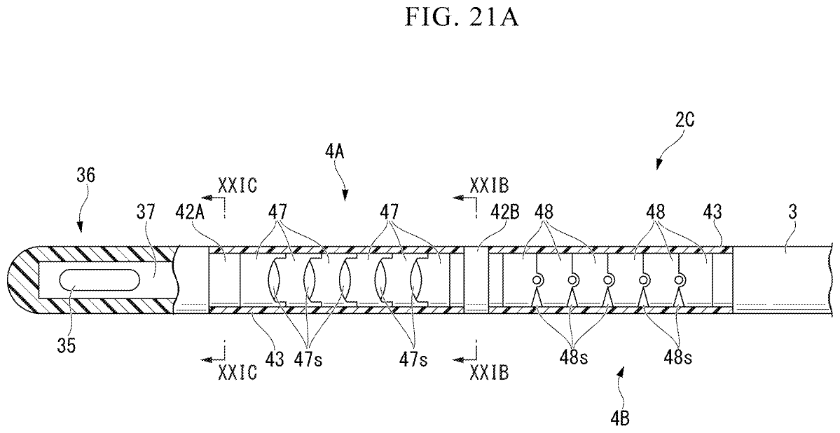

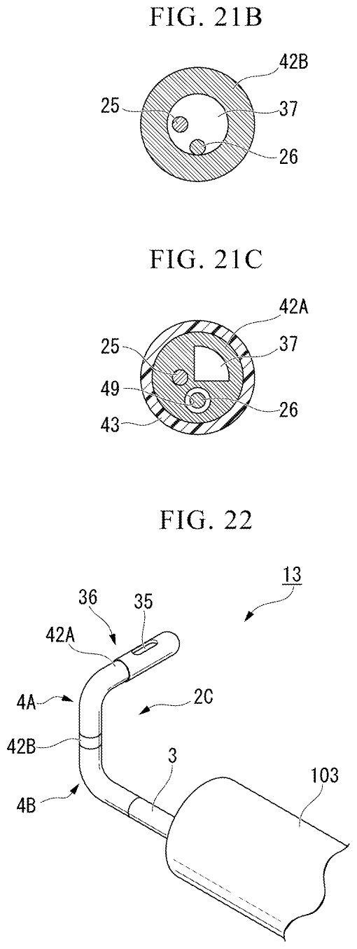

FIG. 21A is a partial cross-sectional view of the distal end portion of the endoscopic treatment tool 13.

As shown in FIG. 21A, the second bending portion 4B is provided at a more distal end side than the distal end portion of the sheath 3 and is connected to the distal end portion of the sheath 3. The first bending portion 4A is provided at a more distal end side than the second wire fixing portion 42B and is connected to the second wire fixing portion 42B. The suction portion 36 is provided at a more distal end side than the first wire fixing portion 42A (wire fixing portion) and is connected to the first wire fixing portion 42A. A suction port 35 is opened at the side of the distal end of the suction portion 36.

The configurations of the first bending portion 4A, the second bending portion 4B, the first wire 25 (wire), and the second wire 26 are the same as those of the second embodiment. However, in the present embodiment, the relationship between the bending direction of the first bending portion 4A and the bending direction of the second bending portion 4B is different, and correspondingly, the positional relationship between the first wire 25 and the second wire 26 in the insertion portion 2C is different. Further, in the present embodiment, the first wire fixing portion 42A is completely covered with the outer layer 43.

In the present embodiment, as shown in FIG. 21A, the gap 47s, which is formed when the plurality of joint pieces 47 in the first bending portion 4A are connected, and the gap 48s, which is formed when the plurality of joint pieces 48 in the second bending portion 4B are connected, are arranged with their phases shifted by 90 degrees around the central axis of the insertion portion 2C (around the axis extending in the longitudinal axis direction of the insertion portion 2C).

FIG. 21B is a cross-sectional view taken along line XXIB-XXIB of FIG. 21A, showing the configuration of the second wire fixing portion 42B. FIG. 21C is a cross-sectional view taken along the line XXIC-XXIC in FIG. 21A, showing the configuration of the first wire fixing portion 42A.

Similar to the second embodiment, the first wire 25 and the second wire 26 have straightness. As shown in FIG. 21C, the first wire 25 is fixed to the first wire fixing portion 42A. The second wire 26 is fixed to the second wire fixing portion 42B as shown in FIG. 21B. Further, as shown in FIG. 21C, the second wire 26 extends up to the first wire fixing portion 42A, and the distal end thereof is inserted into the insertion hole 49 provided in the first wire fixing portion 42A so as to be capable of advancing and retracting.

In the present embodiment, the gap 47s and the gap 48s are arranged with their phases shifted by 90 degrees around the central axis of the insertion portion 2C. The first wire 25 is arranged on the side where the gap 47s is formed, and the second wire 26 is arranged on the side where the gap 48s is formed. Therefore, the first wire 25 and the second wire 26 are arranged with their phases shifted by 90 degrees around the central axis of the insertion portion 2C (around the axis line extending in the longitudinal axis direction of the insertion portion 2C). That is, the second wire 26 is arranged with a phase shift of 90 degrees around the central axis of the second bending portion 4B (around the axis extending in the longitudinal axis direction of the second bending portion 4B) with respect to the first wire 25.

As shown in FIG. 21A, the suction port 35 of the suction portion 36 is provided at a position facing the gap 47s in the first bending portion 4A.

As shown in FIGS. 21A to 21C, in the insertion portion 2C, a conduit 37 that connects the suction portion 36, the first bending portion 4A, and the second bending portion 4B is formed. The conduit 37 communicates with the outside through the suction port 35 of the suction portion 36. The conduit 37 is connected to the third operation portion 7C (see FIG. 20) and is connected to a suction device (not shown) via the suction tube 81.

The bending operation of the endoscopic treatment tool 13 of the present embodiment configured as described above will be described with reference to FIG. 22.

FIG. 22 is a view showing the distal end portion of the endoscope treatment tool 13 in a state in which the first bending portion 4A and the second bending portion 4B of the endoscope treatment tool 13 are bent.

In the example shown in FIG. 22, the endoscopic treatment tool 13 is arranged to be inserted in the treatment tool insertion channel 105 (see FIG. 17A) of the endoscope 103 such that the suction port 35 of the suction portion 36 faces upward in a state in which both the first bending portion 4A and the second bending portion 4B are bent. More specifically, the endoscope treatment tool 13 is arranged so that the second bending portion 4B bends upward and the first bending portion 4A bends rightward when viewed from the endoscope 103 side.

The bending of the first bending portion 4A to the right when viewed from the endoscope 103 side means that the first bending portion 4A bends so that its distal end portion is directed to the right when viewed from the endoscope 103 side.

In the present embodiment, the gap 47s and the gap 48s (the first wire 25 and the second wire 26) are arranged with their phases shifted by 90 degrees around the central axis of the insertion portion 2C, so that the first bending portion 4A can be bent rightward when viewed from the endoscope 103 side, and the second bending portion 4B can be bent upward. Further, since the suction port 35 of the suction portion 36 is provided at a position facing the gap 47s in the first bending portion 4A, in a state in which both the first bending portion 4A and the second bending portion 4B of the endoscopic treatment tool 13 are bent, the suction port 35 of the suction portion 36 can face upward as shown in FIG. 22.

Also in the present embodiment, since the first wire 25 has straightness, when returning the first bending portion 4A to the straight state before bending, the bending of the first bending portion 4A is reliably released (straightened) due to the straightness of the first wire 25. Similarly, since the second wire 26 has straightness, when returning the second bending portion 4B to the straight state before bending, the bending of the second bending portion 4B is reliably released (straightened) due to the straightness of the second wire 26.

Further, also in the present embodiment, both the first wire 25 and the second wire 26 extend from the first wire fixing portion 42A through the inside of the first bending portion 4A, the inside of the second bending portion 4B, and the inside of the sheath 3. Thereby, the first bending portion 4A and the second bending portion 4B have substantially the same contents, and the bending rigidity of the first bending portion 4A and the bending rigidity of the second bending portion 4B can be made substantially the same.

As described above, also in the endoscopic treatment tool 13 (medical device) of the present embodiment, as in the second embodiment, even in a case in which a plurality of bending portions are provided, the contents of the endoscopic treatment tool 13 can be reduced, and the diameter thereof can be reduced.

Further, also in the endoscopic treatment tool 13 (medical device) of the present embodiment, since the first wire 25 and the second wire 26 have straightness, the bending of the bending portion is reliably released (straightened) due to the straightness of the first wire 25 and the second wire 26.

Furthermore, also in the endoscopic treatment tool 13 (medical device) of the present embodiment, the bending rigidity of the first bending portion 4A and the second bending portion 4B is substantially the same, and therefore, the release (straightening) of the bending of the first bending portion 4A and the second bending portion 4B can be controlled more preferably.

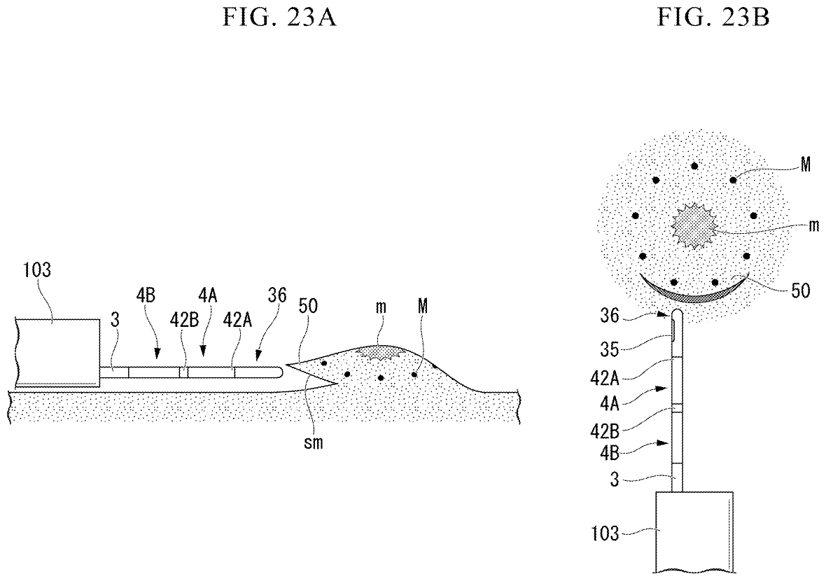

Next, referring to FIGS. 23A to 27B, a second procedure that uses the endoscopic treatment tool 12 (medical device) of the third embodiment and the endoscopic treatment tool 13 (medical device) of the fourth embodiment will be described. The second procedure is different from the first procedure at least in that the endoscopic treatment tool 13 (mucous membrane lifting tool) of the fourth embodiment is used instead of the endoscopic treatment tool 11 (grasping forceps) of the second embodiment.

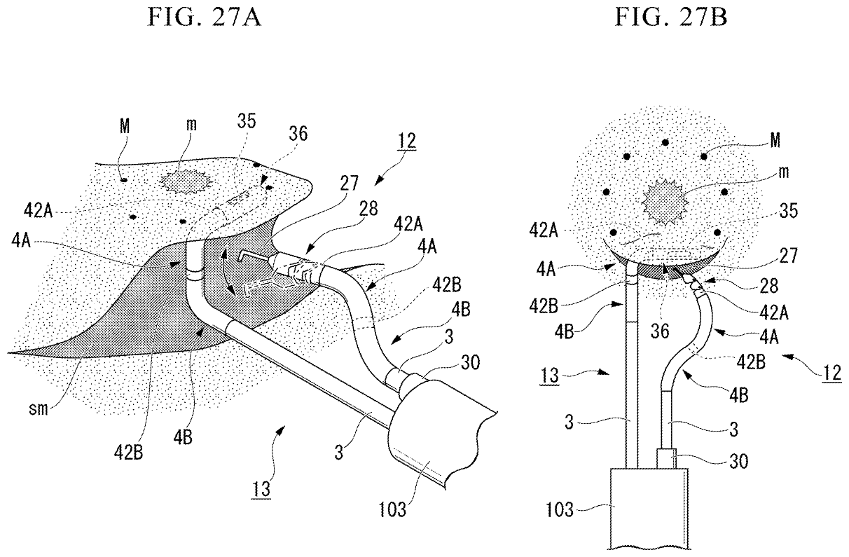

FIGS. 23A to 27B are schematic views showing a process of the second procedure. FIGS. 23A, 24A, 25A, 26A and 27A show a side view of a process of the procedure, and FIGS. 23B, 24B, 25B, 26B and 27B show a plan view of the process of the procedure. Further, FIG. 27A shows a perspective view of a process of the procedure, and FIG. 27B shows a plan view of the process of the procedure. The processes of the procedure shown in FIGS. 23A, 24A, 25A, 26A and 27A are the same, and the processes of the procedure shown in FIGS. 23B, 24B, 25B, 26B and 27B are the same.

As shown in FIGS. 23A and 23B, in the same manner as the first procedure, the marking M is made on the mucous membrane of the digestive tract having the lesion m, and an initial incision is made on the mucous membrane around the lesion m, so that an incised mucous membrane 50 is formed. Then, the endoscopic treatment tool 13 (mucous membrane lifting tool) is inserted into the treatment tool insertion channel 105 (see FIG. 17A) of the endoscope 103, and the first bending portion 4A (bending portion) and the second bending portion 4B of the endoscope treatment tool 13 are protruded from the endoscope 103 toward the distal end side.

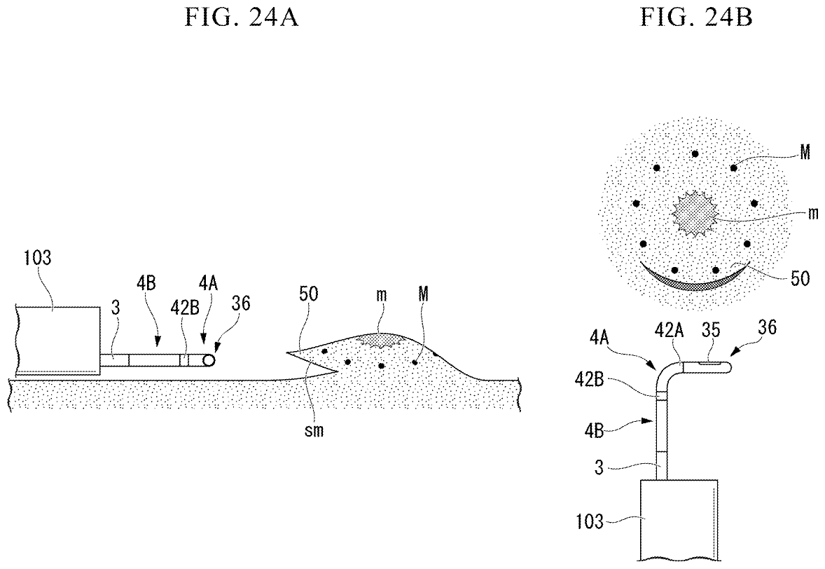

Next, as shown in FIGS. 24A and 24B, the first operation portion 6A (see FIG. 20) of the endoscopic treatment tool 13 is operated to pull the first wire 25 (wire) (see FIG. 21C), and bend the first bending portion 4A rightward when viewed from the endoscope 103 side.

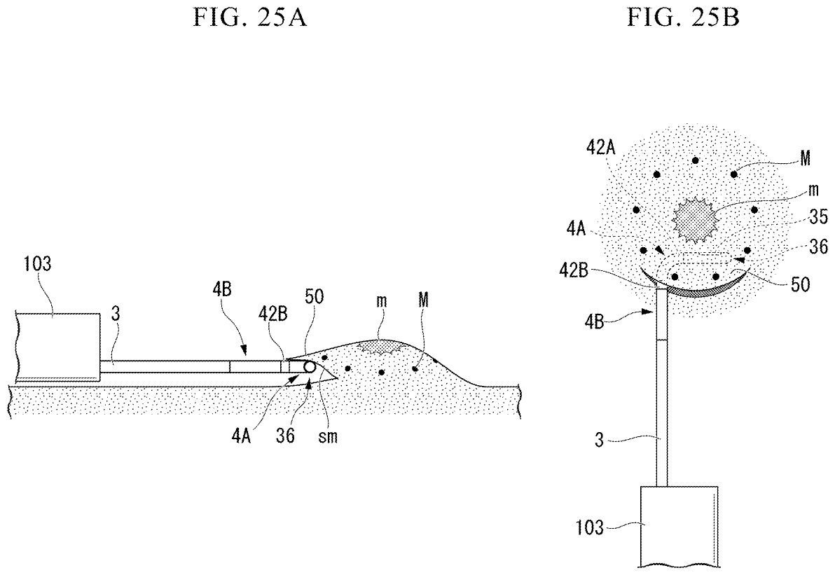

Next, as shown in FIGS. 25A and 25B, the endoscopic treatment tool 13 is pushed toward the distal end side, and the suction portion 36 of the endoscopic treatment tool 13 and the curved first bending portion 4A are also made to slip under the incised mucous membrane 50.

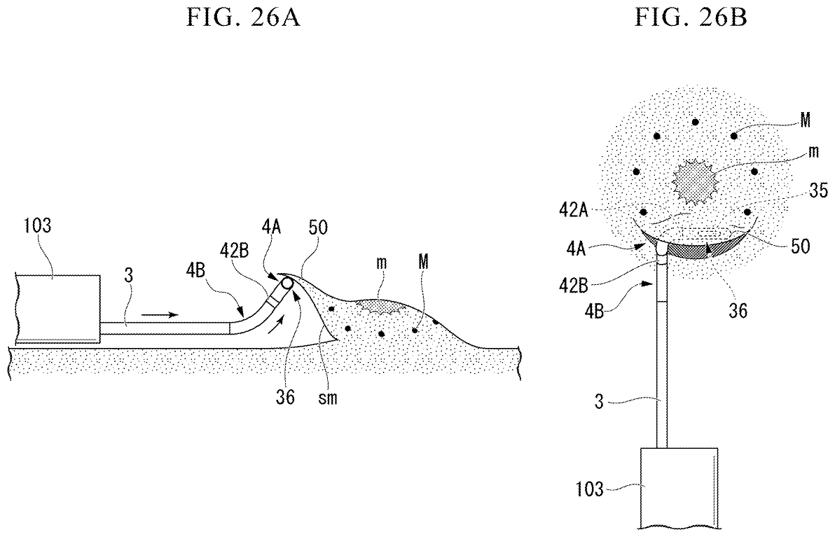

Next, as shown in FIGS. 26A and 26B, the second operation portion 6B (see FIG. 20) of the endoscopic treatment tool 13 is operated while further pushing the endoscopic treatment tool 13 (sheath 3) toward the distal end side, and the second wire 26 (see FIG. 21B) is pulled to bend the second bending portion 4B upward. By this series of operations, the suction portion 36 of the endoscopic treatment tool 13 contacts the incised mucous membrane 50 from below so that the suction port 35 faces the incised mucous membrane 50, and the endoscopic treatment tool 13 can lift up the incised mucous membrane 50. When lifting up the incised mucous membrane 50, a suction device (not shown) is activated. The suction port 35 of the suction portion 36 communicates with the conduit 37 (see FIG. 21A), and the conduit 37 is connected to a suction device via a connecting pipe 72 and a suction tube 81 (see FIG. 20). Therefore, by suctioning the incised mucous membrane 50 from the suction port 35, the incised mucous membrane 50 can be fixed to the suction portion 36. In this state, if the endoscopic treatment tool 13 (sheath 3) is pushed further toward the distal end side, the incised mucous membrane 50 can be further lifted up.

Next, as shown in FIGS. 27A and 27B, in the same manner as the first procedure, the endoscope treatment tool 12 inserted through the treatment tool insertion channel 106 (see FIG. 17A) of the endoscope 103 is protruded from the endoscope 103 toward the distal end side and is curved into a substantially S-shape. The electrode 27 of the endoscopic treatment tool 12 is brought into contact with the submucosal layer sm in a state in which the incised mucous membrane 50 is lifted up by the endoscopic treatment tool 13, and the swing portion 28 is swung and the electrode 27 is swung, so that the submucosal layer sm is incised to excise the mucous membrane including the lesion m.

When the excision of the lesion m is completed by repeatedly incising the submucosal layer sm, the first operation portion 6A and the second operation portion 6B (see FIG. 20) of the endoscopic treatment tool 13 are operated to release the traction by the first wire 25 and the second wire 26 (see FIGS. 21B and 21C), so that the first bending portion 4A and the second bending portion 4B of the endoscopic treatment tool 13 are returned (straightened) to the straight state before bending. Since the first wire 25 and the second wire 26 have straightness, the straightness of the first wire 25 and the second wire 26 reliably releases (straightens) the bending of the first bending portion 4A and the second bending portion 4B.

Similarly, the first operation portion 6A and the second operation portion 6B (see FIG. 14) of the endoscopic treatment tool 12 are operated to release the traction by the first wire 25 and the second wire 26 (see FIG. 15), so that the first bending portion 4A and the second bending portion 4B of the endoscopic treatment tool 12 are returned (straightened) to the straight state before bending. Since the first wire 25 and the second wire 26 have straightness, the straightness of the first wire 25 and the second wire 26 reliably releases (straightens) the bending of the first bending portion 4A and the second bending portion 4B.

Finally, the endoscopic treatment tool 12 and the endoscopic treatment tool 13 are removed from the endoscope 103, and a series of treatments is completed.

As described above, also in the procedure (second procedure) using the endoscopic treatment tool 12 (medical device) and the endoscopic treatment tool 13 (medical device), the first wire 25 and the second wire Since 26 has straightness, the straightness of the first wire 25 and the second wire 26 reliably releases (straightens) the bending of the bending portion, and a smooth procedure can be performed. Further, since the bending rigidity of the first bending portion 4A and the second bending portion 4B are substantially the same, the release (straightening) of the bending of the first bending portion 4A and the second bending portion 4B can be controlled more preferably to carry out the procedure.

Although the respective embodiments of the present invention have been described above, the technical scope of the present invention is not limited to the above-described embodiments and modifications, the combination of components may be changed, and various changes can be added to or deleted from each component, without departing from the spirit of the present invention.

For example, in each of the above-described embodiments, an endoscopic treatment tool has been described as an example of a medical device that is operated by bending with a wire having straightness, but a medical device which is bent by a straight wire may be, for example, an endoscope. In an endoscope including a bending portion capable of bending, the bending portion may be operated with a wire having straightness. In this case, since one bending portion is operated with one wire, the bending portion of the endoscope can be thinned. Further, since the wire that operates the bending portion of the endoscope has straightness, the bending of the bending portion of the endoscope is reliably released (straightened) due to the straightness of the wire.

Further, in the above-described second embodiment and third embodiment, an example in which a straight wire is used as the first wire 25 and the second wire 26 has been described, but the forceps portion operating wire 24 and the electrode swing wire 34 may be formed by wires having straightness. In this case, the forceps portion 22 and the swing portion 28 can be controlled and operated more preferably. Of course, a part of the forceps portion operating wire 24 and the electrode swing wire 34 may be formed by a straight wire member having straightness.

Further, as a modification of the wire configuration in the above-described second embodiment, an example in which the first wire 25A is composed of the straight wire member 25a having straightness and the non-straight wire member 25b, and the second wire 26A is composed of the straight wire member 26a having straightness and the non-straight wire member 26b has been described, but only one of the first wire (wire) and the second wire may be composed of a straight wire member having straightness and a non-straight wire member. For example, the first wire may be composed of a straight wire member and a non-straight wire member, and the second wire may be composed of only a straight wire member. The configuration of the wire can be flexibly selected according to the bending performance required for the bending portion to be operated.

Further, in the above-described second embodiment, the first bending portion 4A (bending portion) has a plurality of joint pieces 47 swingably connected to each other, and the second bending portion 4B has a plurality of joint pieces 48 swingably connected to each other, but one of the first bending portion 4A and the second bending portion 4B may be configured by a joint piece, and the other may be, for example, a configuration (configuration shown in FIGS. 8A and 8B) according to a modification of the configuration of the bending portion in the first embodiment. The configuration of the bending portion can be flexibly selected according to the bending performance required for the bending portion to be operated.

Further, in the above-described second procedure, an example in which the incised mucous membrane 50 is fixed by suction has been described, but the fixing method is not limited to suction, and, for example, a grasping mechanism, a locking mechanism, or the like may be provided at the distal end of the endoscopic treatment tool, and the incised mucous membrane 50 may be fixed by grasping, locking, or the like. An appropriate fixing method can be adopted depending on the situation of the site to be treated.

In each of the above-described embodiments, a case where the number of bending portions of each endoscope treatment tool is one or two has been described as an example, but the number of bending portions of each endoscope treatment tool may be three or more. In a case in which there are a plurality of bending portions, the bending directions of the bending portions can be freely combined. Even in such a case, by bending each bending portion and releasing the bending with a single wire having straightness, the diameter of the insertion portion (bending portion) of the medical device can be reduced, and it is possible to reliably release (straighten) the bending of the bending portion.

Further, in each of the above-described embodiments, the catheter, the grasping forceps, the high-frequency knife, and the mucous membrane lifting tool have been described as examples of the endoscopic treatment tool, but a medical device such as an endoscopic treatment tool to which the present invention is applied can be appropriately selected according to the application.

According to the medical device of the above-described embodiment, bending of a bending portion and release of bending (straightening) can be realized by a single wire, and it is possible to reliably release (straighten) bending of a bending portion.

What is claimed is:

1. A medical device comprising:
a sheath;
a first bending portion provided at a distal end side of the sheath;
a second bending portion provided between the first bending portion and the sheath;
a first wire fixed to a first wire fixing portion provided at a distal end side of the first bending portion and inserted in the sheath toward a proximal end side of the sheath; and
a second wire fixed to a second wire fixing portion provided between the first bending portion and the second bending portion and inserted in the sheath toward the proximal end side of the sheath,
wherein a part of the first wire positioned in the first bending portion and a part of the first wire positioned in the second bending portion have straightness, and
at least a part of the second wire positioned in the second bending portion has straightness.

2. The medical device according to claim 1, wherein
the first wire and the second wire are composed of
a straight wire member having straightness, and
a non-straight wire member made of a material different from that of the straight wire member, and
at least a part of the first wire positioned in the first bending portion and a part of the first wire positioned in the second bending portion are composed of the straight wire member, and
at least a part of the second wire positioned in the second bending portion is composed of the straight wire member.

3. The medical device according to claim 1, wherein the first bending portion and the second bending portion have lower bending rigidity than the first wire fixing portion, the second wire fixing portion, and the sheath.

4. The medical device according to claim 1, wherein the second wire is arranged with a phase shift of 180 degrees around a central axis of the second bending portion with respect to the first wire.

5. The medical device according to claim 1, wherein the second wire is arranged with a phase shift of 90 degrees around a central axis of the second bending portion with respect to the first wire.

6. The medical device according to claim 1, wherein the second wire fixing portion further includes an insertion hole through which the first wire is inserted so as to be capable of moving forward and backward.
